(19) United States
(12) Patent Application Publication
(10) Pub. No.: US 2026/0240428 A1
(43) Pub. Date: Aug. 20, 2026

(54) Method and System for Predictive Analytics of the Quality of Vision

(71) Applicant: Tracey Technologies, Corp., Houston, TX (US)

(72) Inventors: Youssef S. Wakil, Houston, TX (US); Vasyl Molebny, Houston, TX (US); Ioannis Pallikaris, Maleviziou, Crete (GR)

(73) Assignee: Tracey Technologies, Corp., Houston, TX (US)

(21) Appl. No.: 19/643,509

(22) Filed: Apr. 9, 2026

Related U.S. Application Data
(63) Continuation-in-part of application No. 19/377,291, filed on Nov. 3, 2025.
Application No. 19/377,291 is a continuation-in-part of application No. PCT/US2024/027895, filed on May 4, 2024.
(60) Provisional application No. 63/500,255, filed on May 4, 2023.

(51) Int. Cl. A61B 3/00; A61B 3/10

(52) U.S. Cl. CPC A61B 3/0025; A61B 3/10

(57) ABSTRACT

The present invention is a predictive analytics method and system for the optimization of vision quality in a subject which uses at least one of an optical quality index of a total eye, an optical quality index of a cornea or a dysfunctional lens index as preoperative conditions input. The output is a type and at least one outcome of a procedure performed on an eye of the patient. A correlation between measured data of preoperative conditions from the cloud library and the input data of preoperative conditions for each component of pre-operative conditions is calculated to enable recommendations by a doctor.

### CROSS-REFERENCE TO RELATED APPLICATIONS

This continuation-in-part application claims benefit of priority under 35 U.S.C. § 120 of pending patent application U.S. Ser. No. 19/377,291, filed Nov. 3, 2025, which claims benefit of priority under 35 U.S.C. § 120 and § 365(c) of international patent application PCT/US2024/027895, filed May 4, 2024, now abandoned, which claims benefit of priority under 35 U.S.C. § 119(e) of provisional patent application U.S. Ser. No. 63/500,255, filed May 4, 2023, now abandoned, the entirety of all of which are hereby incorporated.

### BACKGROUND OF THE INVENTION

### Field of the Invention

The present invention relates to the fields of ophthalmology and analytic tools useful in ophthalmologic medical procedures. More particularly, the present invention is an ophthalmologic means used for eye examination and treatment, especially for planning medical procedures on the eye, particularly for planning an eye surgery, that are based on comparative analytics of measured optometric characteristics considering the experience of other similar cases with the machine learning procedures and final choice based on maximization of indices empirically adjusted to the decimal range, that are the quality of vision index (QVI™), or/and the cornea performance index (CPI™), or/and the dysfunction lens index (DLI™).

### Description of the Related Art

Replacement of the cataractous crystalline lens with the intraocular implant at the end of 40-s by H. Ridley stimulated the search for adequate description of the quality of human vision. F. W. Cambell and R. W. Gubisch in their pioneering studies of the optical quality of the human eye. (J. Physiol., 1966, 186, pp. 558-578) used the modulation transfer function and its Fourier transform, that is a linespread function.

J. Liang and D. R. Williams paid attention to the role of higher-order aberrations on the retinal image quality (J. Opt. Soc. Am. A, 1997, 11, pp. 2873-2883). In the U.S. Pat. No. 7,357,509, D. R. Williams, et al. proposed several metrics to predict the subjective impact of the eye's wavefront aberrations based on wavefront errors or slopes, the area of the critical pupil, a curvature parameter, the point spread function, the optical transfer function, or the like. Other techniques include the fitting of a sphero-cylindrical surface, the use of multivariate metrics, and customization of the metric for patient characteristics such as age.

These metrics are insufficient for making prediction for the success of the planned surgery. Parameters of the replacing intraocular lenses and the technologies used for implantation are not taken into account. The experience of similar cases also is to be included into the planning schedule.

In U.S. Pat. No. 8,388,137, Dreher and Lai proposed predicting the post-operational visual acuity from the statistics of the plurality of measured wavefront aberrations associated with the measured visual acuity. The drawback of this approach is that there is no feedback enabling the perfectioning of the prediction.

Angelides (U.S. Pat. No. 10,199,126) proposed individualized feedback via the internet for health improvement plans for an individual participant facing a chronic disease or consistent discomfort. Its principle consists in storing the current information on the status of the patient and getting continuously the advising. In a system for surgical planning (U.S. Pat. No. 11,672,419), Dewey et al. use a corneal topography subsystem, an axis determining subsystem, an OCT-based ranging subsystem, and a refraction measurement subsystem, enabling modification of planning in the result of refraction measurement.

In case of planning an ophthalmologic surgery, a doctor needs to use stored experience and data purposely acquired. Adequate for clinical usage is empirical indexing of the quality of vision based on quality vision index QVI, cornea performance index CPI, and dysfunction lens index DLI, calculated from measured aberrations that take into account empirically confirmed inter-aberration interaction of higher-order aberrations of the cornea and of the crystalline lens (F. Faria-Correia, et al. J Refract Surg. 2016, 32, pp. 244-248).

Thus, there is a recognized need in the art for an improved means for determining visual corrections and planning a surgery based on the same. Particularly, the prior art is deficient in a predictive analytics tool that utilizes descriptive components of a quality vision index (QVI), a cornea performance index (CPI), and a dysfunction lens index (DLI) pre-operationally and post-operationally. The present invention fulfills this long-standing need and desire in the art.

### SUMMARY OF THE INVENTION

The invention is directed to a method for predictive analytics of the quality of vision of a patient prior to a medical procedure. In the method the optometric characteristics of the total eye and its components and spatial distributions and time variations thereof are measured pre-operationally. A pre-operational digital twin is built based on measured optometric characteristics, where the pre-operational digital twin consists of a modifiable part and of a non-modifiable part. A pre-operational vector space is built from the modifiable part of the pre-operational digital twin and each vector therein is provided with a correctable weight coefficient. A scoring metrics of the quality of vision is built pre-operationally and is represented by a quality of vision index (QVI), a cornea performance index (CPI), and a dysfunction lens index (DLI), where each index contains related descriptors consisting of modifiable parts and non-modifiable parts of the pre-operational digital twin. A comparative analysis of the pre-operational vector space with post-operational vector spaces acquired in the same medical procedure of other patients stored in a database is performed, where the comparative analysis defines a best fit vector space for the medical procedure. Scoring metrics of the quality of vision is calculated based on measured and predicted data, where the scoring metrics are represented by the quality of vision index (QVI), the cornea performance index (CPI), and the dysfunction lens index (DLI). Correctable weight coefficients of the best fit vector space are modified to maximize related scoring metrics and to present it as a predicted score for the medical procedure. The pre-operational digital twin is reconstructed into a predicted digital twin by combining the non-modifiable part of the pre-operational digital twin and the modified part of the digital twin derived from the best fit vector space after its modification by the correctable weight coefficients.

The invention is directed to a related method further comprising storing the pre-operational digital twin and the predicted digital twin and displaying predicted data in a scoring metrics format and in a digital twin format.

The invention is directed to a another related method further comprising repeating post-operationally the measuring step and the building steps, performing post-operationally a comparative analysis of the predicted and of the current post-operational vector spaces in a machine-learning procedure correcting the weight coefficients for the same medical procedures in the future and displaying the results of post-operational machine-learning comparative analysis, storing the results of post-operational machine-learning comparative analysis.

The invention also is directed to a system for predictive analytics of the quality of vision. A sensor subsystem contains a first sensor, a second sensor and a third sensor configured to measure, separately or in combination, optometric characteristics of a total eye and its components, their spatial distributions and time variations. A storage subsystem consists of a preoperational memory, an archived-cases memory, and a dynamic interrogation switch, where the preoperational memory and the archived-cases memory consists of per-patient cells each of which has a preoperational sub-cell, a prediction sub-cell, and a post-operational sub-cell, and each of the per-patient cell in the archived-cases memory is connected to the dynamic interrogation switch. A control subsystem contains a control signals generator, an interface, and a display, where the display is connected to the decision-making subsystem through the interface and the control signals generator connected to the interface. A decision-making subsystem consists of a twin builder, a first metrics builder, a second metrics builder, a first builder of vector space, a second builder of vector space, a processing unit with a first input and a second input, and a weight coefficients unit, where the first builder of vector space is connected to the first input, the second builder of vector space is connected to the second input and outputs of the first metrics builder and of the second metrics builder are connected to the display. An intra-system communication bus is configured to exchange signals, commands and data among the sensor subsystem, the decision-making subsystem, the storage subsystem, and the control system pre-operationally and post-operationally.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the above-recited features of the invention are to be understood in detail, more particular descriptions of the invention briefly summarized above are illustrated in the appended drawings. These drawings form a part of the specification. However, that the appended drawings illustrate preferred embodiments of the invention, they are not to be considered limiting in their scope.

FIG. 1 is a general overview of the electronic components comprising the predictive analytics tool.

FIG. 2 illustrates a reduced visual function via the Dysfunction Lens Index (DLI).

FIGS. 3A-3D illustrate stages I (FIG. 3A), II (FIG. 3B), III (FIG. 3C), a spectrum of states I, II and III (FIG. 3D) of a cataract via the Dysfunction Lens Index.

FIG. 4 illustrates the angle alpha and angle kappa distances in the eye.

FIGS. 5A-5B illustrate optical alignment measurements in a subject's eye with a low angle alpha (FIG. 5A) and an extremely high angle alpha (FIG. 5B).

FIGS. 6A-6D illustrate an example of a corneal performance result (FIG. 6A), a corneal axial map (FIG. 6B), corneal performance at various radii (FIG. 6C), and corneal performance scores (FIG. 6D).

FIG. 7 illustrates an example of a total optical performance result.

FIGS. 8A-8H illustrate lens pathologies in three subjects left (OS) eyes (FIGS. 8A-8B, 8E-8F and right (OD) eyes (FIGS. 8C-8D, 8G-8H) pre-operative (FIGS. 8A, 8C, 8E, 8G) and postoperative (FIGS. 8B, 8D, 8F, 8H) surgery.

FIGS. 9A-9C illustrate corneal pathologies in three subjects.

FIGS. 10A-10C illustrate postoperative problems.

FIGS. 11A-11B show population statistics for CPI performance with an absolute count (FIG. 11A) and relative frequency (FIG. 11B).

FIGS. 12A-12C show population statistics for Quality of Vision (QVI) performance with an absolute count (FIG. 12A), relative frequency (FIG. 12B) and the distribution of QVI scores for a population for each of the 4^{th} to 10^{th} age decile (FIG. 12C).

FIGS. 13A-13D show the distribution of scores for each of postoperative, preoperative and no surgery populations for the Corneal Performance Index (CPI) (FIG. 13A), the Dysfunction Lens Index (DLI) (FIGS. 13B-13C) and Quality of Vision Index (QVI) (FIG. 13D).

FIGS. 14A-14C are Placido images illustrating initial rings (FIG. 14A), loss of sharpness (FIG. 14B) and discontinuity (FIG. 14C).

FIGS. 15A-15B illustrate Tear Film Analysis of a normal tear film (FIG. 15A) and a dry eye (FIG. 15B) and a Tear Film Analysis display (FIG. 15C).

FIG. 16 demonstrates the step-by-step solution of the prediction problem. It has two stages: pre-operational analysis of the current case (steps 10a-10j) and post-operational analysis of the outcome with machine-learning to be better prepared for the future cases (steps 10k-10m).

FIG. 17 illustrates the origins of initial ray tracing parameters, laser beam positions at the aperture of the eye, coordinates of laser spots on the retina, and the signals received by the detectors,.

FIG. 18 illustrates the distribution of refraction over the entrance aperture and displayed values of refraction.

FIG. 19 Illustrates of the corneal part of the refraction acquired with a topographer: Placido disk reflected from the cornea, distribution of refraction, and the digitized description of the corneal topography.

FIG. 20 shows the beam projection points in the plane of the cornea.

FIG. 21 defines the nodes of reference mesh as coordinates of beam projection points in the plane of the cornea.

FIG. 22 is a reference mesh configured from the nodes corresponding to the beam projection points in the plane of the cornea.

FIG. 23 shows the beam projection spots in the plane of the retina.

FIG. 24 shows the coordinates of the beam projection in the plane of the retina to be used as correction data for creating the comparison mesh.

FIG. 25 illustrates the created configuration of the comparison mesh.

FIG. 26 shows the histogram of refraction distortion in the eye. Horizontal axis corresponds to the degree of distortion of cells in the comparison mesh. Vertical axis corresponds to the number of cells. Refraction quality index is calculated as the ration of the number of “bad” cells to the total number of cells of the mesh.

FIG. 27 represents the map of refraction quality of the eye of the patient in FIGS. 21-27 which relate to the case of low RQI (pre-operational examination).

FIG. 28 shows the comparison mesh of the same patient as in FIGS. 21-27 post-operationally.

FIG. 29 shows the histogram of refraction distortion in the eye of the same patient as in FIGS. 21-27 post-operationally.

FIG. 30 represents the map of refraction quality of the eye of the same patient as in FIGS. 21-27 post-operationally.

FIG. 31 shows an example of the polynomial description of the total eye as a screenshot of the ITRACE (Tracey Technologies Corp.) display.

FIG. 32 demonstrates the modulation transfer function (MTF) derived from the polynomial description presented in FIG. 31. The data are used for QVI calculations.

FIG. 33 shows an example of the polynomial description of the cornea as a screenshot of the ITRACE display (the same eye as in FIG. 31).

FIG. 34 demonstrates the modulation transfer function (MTF) derived from the polynomial description presented in FIG. 33. The data are used for CPI calculations.

FIG. 35 is an example of a screenshot of the ITRACE display demonstrating the metrics of the eye with low vision (CPI=3.9, DLI=3.6, QVI=1.7).

FIG. 36 is another example of a screenshot of the ITRACE display demonstrating the metrics of the eye with good vision (CPI=7.7, DLI=10.0, QVI=10.0).

FIG. 37 shows a functional schematic of a version of the system of predictive analytics of the quality of vision implementing the teachings of the proposed method in its preoperational configuration. Its main component is a decision-making sub-system accompanied by sensor, control and storage sub-systems.

FIG. 38 is the same functional schematic as in FIG. 37 configured for post-operational analysis. The results are stored in a cell that changes its address from pre-operational to archived-cases storage being accessible for subsequent cases.

FIG. 39 shows the cell 300.(n+1) in the pre-operational storage.

FIG. 40 demonstrates shifting the cell 300.(n+1) with the data of digital twins of the current case from the pre-operational storage to the archived-cases storage.

FIG. 41 is a functional diagram of a version of the sensor subsystem with integrated optics having a common optical axis.

FIG. 42 presents a version of the proposed system, where the role of data bus is exercised by the internet connection through modems at client and cloud sites. The decision-making sub-system is located in the cloud.

FIG. 43 presents a version like that shown in FIG. 42 but with the decision-making sub-system located at the client site.

FIG. 44 illustrates multiple proposed systems in the version of decision-making sub-system common for all clients of a net configuration located in the cloud.

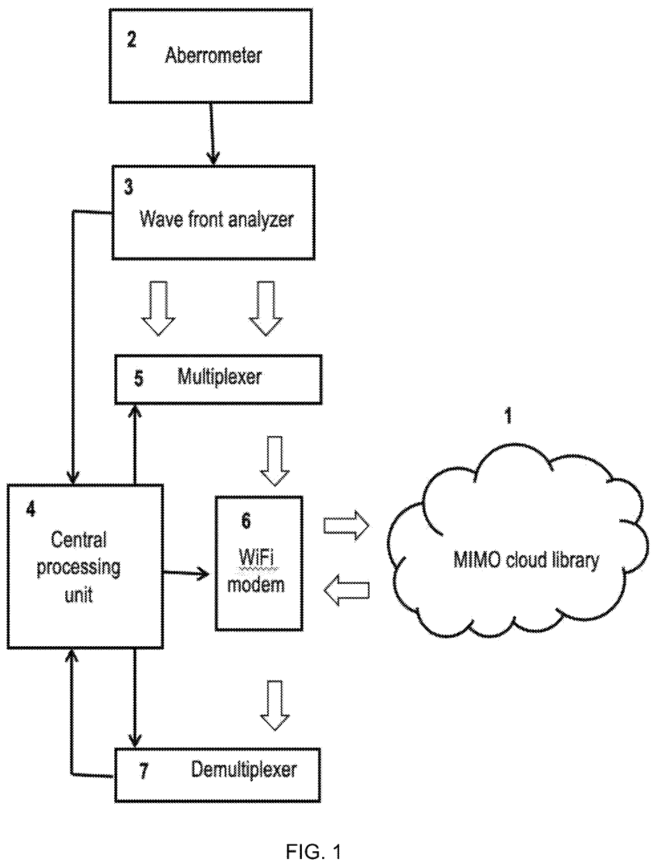

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, the terms “comprise” and “comprising” are used in the inclusive, open sense, meaning that additional elements may be included.

As used herein, the terms “consist of” and “consisting of” are used in the exclusive, closed sense, meaning that additional elements may not be included.

As used herein, the conditional language, such as, among others, “can”, “might”, “may”, “e.g.”, “for example”, and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

As used herein, “patient” and “subject” are used interchangeably.

In one embodiment of the invention there is provided a predictive analytics tool for vision quality optimization in a subject, comprising a big data multiple input—multiple output (MIMO) cloud library with password access for authorized users thereof, the multiple input data containing at least one component of preoperative conditions and the multiple output data containing a type and at least one outcome of a technology performed on an eye of the subject.

In this embodiment, the at least one component may comprise an optical quality index of a total eye, an optical quality index of a cornea or a dysfunctional lens index. Also in this embodiment the multiple input data of preoperative conditions may be measured for the subject to be treated. In addition, a correlation may be calculated between measured data of preoperative conditions and the multiple input data of preoperative conditions from the cloud library for each component of pre-op conditions. Furthermore at least one case with a highest correlation of preoperative conditions may be selected from the cloud library, and a ranking thereof may be defined in a multi-dimensional vector space for each type and the at least one outcome for the technology performed within the multiple output data from the cloud library. Further still a list of recommended technologies may be output for a final approval by a doctor.

In another embodiment of the present invention there is provided a method for predictive analytics of the quality of vision of a patient prior to a medical procedure, comprising measuring pre-operationally the optometric characteristics of the total eye and its components and spatial distributions and time variations thereof; building a pre-operational digital twin based on measured optometric characteristics, the pre-operational digital twin consisting of a modifiable part and of a non-modifiable part; building a pre-operational vector space from the modifiable part of the pre-operational digital twin and providing each vector therein with a correctable weight coefficient; building pre-operationally a scoring metrics of the quality of vision represented by a quality of vision index (QVI), a cornea performance index (CPI), and a dysfunction lens index (DLI), each index containing related descriptors consisting of modifiable parts and non-modifiable parts of the pre-operational digital twin; performing a comparative analysis of the pre-operational vector space with post-operational vector spaces acquired in the same medical procedure of other patients stored in a database, said comparative analysis defining a best fit vector space for the medical procedure; calculating scoring metrics of the quality of vision based on measured and predicted data, the scoring metrics represented by the quality of vision index (QVI), the cornea performance index (CPI), and the dysfunction lens index (DLI); modifying correctable weight coefficients of the best fit vector space to maximize related scoring metrics and presenting it as a predicted score for the medical procedure; and reconstructing the pre-operational digital twin into a predicted digital twin by combining the non-modifiable part of the pre-operational digital twin and the modified part of the digital twin derived from the best fit vector space after its modification by the correctable weight coefficients.

Further to this embodiment the method comprises storing the pre-operational digital twin and the predicted digital twin; and displaying predicted data in a scoring metrics format and in a digital twin format. In another further embodiment the method may comprise repeating post-operationally the measuring step and the building steps; performing post-operationally a comparative analysis of the predicted and of the current post-operational vector spaces in a machine-learning procedure correcting the weight coefficients for the same medical procedures in the future, and displaying the results of post-operational machine-learning comparative analysis, storing the results of post-operational machine-learning comparative analysis. Particularly, in this further embodiment the machine learning may be based on k-nearest neighbours (kNN) or on neural networking.

In all embodiments calculating a refraction quality index (RQI) may be based on a comparison of a reference mesh of nodes created from coordinates of beam-projected points at an eye aperture with a mesh of nodes representing the coordinates of the beam-projection points in a plane of the eye aperture modified by shifts in a plane of a retina by a refraction state in the beam-projection points, comprising a) measuring a shape distortion of each cell of the mesh of nodes, b) identifying each cell by a degree of distortion, c) building a histogram of distortion spectrum, d) defining a distortion reference threshold for the RQI calculation, and e) calculating an RQI as a number of over-threshold shape-distorted cells to a total number of cells in the mesh.

In an aspect of all embodiments calculating the quality of vision index (QVI) to score a quality of vision of the total eye may be based on an impact of higher order aberrations on a point spread function of the total eye under standard measurement conditions with an empirically corrected individual impact of each of the higher order aberrations. In another aspect of all embodiments calculating the cornea performance index (CPI) to score the corneal fraction in the quality of vision may be based on an analysis of a wave front reconstructed from a corneal topography and conditions of tear film dynamics. In yet another aspect calculating the dysfunction lens index DLI to score the quality of the internal optics of an eye may be based on a contribution of higher order aberrations measured for the internal optics either by substracting a wave front calculated for a cornea from a wave front of a total eye, or by measuring separately a fraction of the aberrations produced by the internal optics.

In all embodiments and aspects thereof the quality of vision index (QVI), the cornea performance index (CPI) and the dysfunction lens index (DLI) have a decimal 0 to 10 scale, where the lowest quality is scored by 0, and the highest quality is scored by 10. Also in all embodiments and aspects thereof the optometric characteristics may comprise a refractive part measured by ray tracing and a structural part measured by optical coherence tomography.

In yet another embodiment of the present invention there is a provided a system for predictive analytics of the quality of vision, comprising a sensor subsystem containing a first sensor, a second sensor and a third sensor configured to measure, separately or in combination, optometric characteristics of a total eye and its components, their spatial distributions and time variations; a storage subsystem consisting of a preoperational memory, an archived-cases memory, and a dynamic interrogation switch, the preoperational memory and the archived-cases memory consisting of per-patient cells each of which has a preoperational sub-cell, a prediction sub-cell, and a post-operational sub-cell, and each of the per-patient cells in the archived-cases memory connected to the dynamic interrogation switch; a decision-making subsystem consisting of a twin builder, a first metrics builder, a second metrics builder, a first builder of vector space, a second builder of vector space, a processing unit with a first input and a second input, and a weight coefficients unit, said first builder of vector space connected to the first input, said second builder of vector space connected to the second input and outputs of said first metrics builder and of said second metrics builder are connected to a display; and a control subsystem containing a control signals generator, an interface, and the display, the display connected to the decision-making subsystem through the interface and the control signals generator connected to the interface; and an intra-system communication bus configured to exchange signals, commands and data among the sensor subsystem, the decision-making subsystem, the storage subsystem, and the control system pre-operationally and post-operationally.

In one aspect of this embodiment, pre-operationally, in the decision-making subsystem a) the twin builder is configured to: generate a pre-operational digital twin of vision in an eye of a patient by integration of data from the sensor subsystem, said pre-operational digital twin consisting of a first part not modified by a medical procedure and a second part subject to modification by the medical procedure; deliver both the first part of the pre-operational digital twin and the second part of the pre-operational digital twin to the first metrics builder and to the storage subsystem; and deliver the second part of the pre-operational digital twin to the first builder of vector space; b) the first metrics builder is configured to generate a score for quality of vision from a quality of vision index (QVI), a dysfunction lens index (DLI), and a cornea performance index (CPI), each of the OVI, DLI and CPI indices is configured to integrate current data from the sensor subsystem; c) the second builder of vector space is configured to receive digital twins from the archived-cases memory consecutively in time through the dynamic interrogation switch, the digital twins delivered to the processing unit under control of the intra-system communication bus; d) the processing unit is configured to: analyze vector spaces received from the first builder of vector space and the second builder of vector space and to generate an output for decision making and for machine-human communication and interaction; and deliver a modified part of a predicted digital twin to the second metrics builder and to the storage subsystem, said first part of the predicted digital twin delivered from the twin builder; e) the second metrics builder is configured to generate a score for quality of vision from the quality of vision index (QVI), the dysfunction lens index (DLI), and the cornea performance index (CPI), each of the OVI, DLI and CPI indices is configured to integrate data from an output of the processing unit; and f) a current-case cell with its two clusters filled-in by the preoperational digital twin and the predicted digital twin is configured to be repositioned from the preoperational memory to the archived-cases memory to keep the post-operational sub-cell free for filling-in after a machine-learning step.

In another aspect of this embodiment, post-operationally, in the decision-making subsystem: g) the twin builder is configured to: generate a post-operational digital twin of the vision of the patient by integrating data from the sensor subsystem, the post-operational digital twin consisting of a first part not modified by a medical procedure and a second part modified by the medical procedure; deliver both the first part of the post-operational digital twin and the second part of the post-operational digital twin to the first metrics builder and to the storage subsystem; and deliver the second part of the post-operational digital twin to the first builder of vector space; h) the first metrics builder is configured to generate the score for quality of vision from the quality of vision index (QVI), the dysfunction lens index (DLI), and the cornea performance index (CPI), each of the OVI, DLI and CPI indices is configured to integrate current data from the sensor subsystem; i) the second builder of vector space is configured to receive the digital twins from the archived-cases memory consecutively in time through the dynamic interrogation switch, the digital twins delivered to the processing unit under control of the intra-system communication bus; j) the processing unit is configured to: analyze vector spaces received from the first builder of vector space and the second builder of vector space and to generate the output for decision making and for machine-human communication and interactiondeliver a modified part of a post-operational digital twin to the second metrics builder and to the storage subsystem to fill-in a post-operational cluster of the archived cases cells, said first part of the post-operational digital twin delivered to the second metrics builder from the twin builder; and apply the machine-learning algorithm to correct a weight of each vector in the vector space for the quality of vision prediction for the next patient to minimize a difference between the predicted metrics and the post-operational metrics; and k) the second metrics builder is configured to generate a post-operational score for the quality of vision from the quality of vision index (QVI), the dysfunction lens index (DLI), and the cornea performance index (CPI), each of the OVI, DLI and CPI indices is configured to integrate data from the output of the processing unit. In this aspect the machine-learning algorithm may be applied as part of an artificial intelligence system.

In this embodiment and aspects thereof the output of the second metric builder may be displayable before and after application of the machine-learning algorithm. Also the first sensor may be a wave front aberrometer, the second sensor may be a corneal topographer and the third sensor may be an optical coherence tomograph. In addition the first sensor may be a wave front aberrometer configured for ray tracing to measure synchronously aberrations of the optometric characteristics of: a) aberrations of the total eye and of the cornea, b) intraocular distances, c) tear film dynamics, and d) optical dysfunction of the crystalline lens. Furthermore the first sensor, the second sensor and the third sensor may be integrated optically to have a common optical axis and mechanically as a solid construction.

Also in this embodiment and aspects thereof the sensor subsystem, the control subsystem and the decision-making subsystem may be configured in at least one three-part combination wirelessly connected to the storage subsystem, the storage subsystem residing in the cloud. Particularly, each of the at least one three-part combination has an exchange-communication modem and the storage subsystem has an exchange-synchronization modem configured to control each of the at least one three-part combinations independently of each other with a data flow synchronized by the exchange-synchronization modem. In addition the sensor subsystem and the control subsystem are configured as at least one first two-part combination and the decision-making subsystem and the storage subsystem are configured as a second two-part combination; wherein the at least one first two-part combination is connected wirelessly to the second two-part combination, the second two-part combination residing in the cloud. Particularly, each of the at least one first two-part combination has an exchange-communication modem and the second two-part combination has an exchange-synchronization modem; wherein each of said at least one first two-part combinations is controlled independently of each other with a data flow synchronized by the exchange-synchronization modem.

The present invention is a predictive analytic tool for vision surgery planning. Generally, the tool includes a big data multiple input-multiple output (MIMO) cloud library, an aberrometer with a wave front analyzer, a central processing unit, a multiplexer, a wi-fi modem, and a demultiplexer. On the pre-op stage, based on the aberrometer collected data, the wave front analyzer provides the central processing unit with an index of the optical quality of the eye, an index of the optical quality of the cornea, an index of the lens optical dysfunction, and data on the tear film conditions. The central processing unit, upon getting the requested access to the MIMO cloud library, allows the wi-fi modem to transfer the data from the wave front analyzer to the multiplexer and to upload the data to the cloud library through one of its open multiple inputs. Through one of the open multiple-access cloud library outputs, the central processing unit gets the access to the data stored in the cloud library and chooses the pre-op data maximally correlated with the current patient's data. The requested data are downloaded to the central processing unit through the wi-fi modem and the demultiplexer. Operating interactively through the central processing unit, doctor adjusts the patient's conditions and the goals of the planned surgery to the post-op results, achieved with the surgery techniques, materials and instruments used by the surgeons of a certified level. After the surgery has been made, the post-op results accompanied by the surgery requisites are uploaded to the cloud library.

The invention provided herein utilizes higher order aberration data in the form of an index based on proprietary measures of the cornea's optical plane defined at the cornea's anterior surface including its tear film where the internal optics higher order aberrations is determined by the subtraction of the cornea's to the entire eyes higher order aberrations. Accumulated scores of each of these three indices, the CPI, DLI and QVI in preoperative and postoperative eyes as well as eyes with pre and post corrective means as with contact lenses or spectacles etc. can provide an table of data for future comparison and analysis of patients results. These indices are quantified in a 0-10 numeric value for such comparisons.

Examples of patients visual results at certain points in time either before or after any procedure either surgical or not may provide such tabular raw data in creating a data set given a patients age, sex and other demographics including patients'refractive errors. The quality of vision as denoted by the lack of higher order aberrations or its reduction can now be measured for each component of cornea, internal optics, and total eye at the retinal plane with a difference of the value between pre-and post-procedure. The preoperative and postoperative exams accumulated over thousands if not millions of cases form the raw data to provide a predictive tool in ascertaining the most likely outcome for new patients undergoing similar procedures or receiving specific implants such as, but not limited to, intraocular lenses.

The predictive analytic tool for vision surgery planning consists of a big data multiple input-multiple output (MIMO) cloud library 1, an aberrometer 2, a wave front analyzer 3, a central processing unit 4, a multiplexer 5, a wi-fi modem 6, and a demultiplexer 7. The MMIMO cloud library 1 has multiple inputs for receiving the information and data from qualified clients and multiple outputs for sending them the information stored in its big database. The pre-op data of the current patient are acquired by the aberrometer 2 and properly processed by its wave front analyzer 3, both usually being constructively in a common housing, such as the iTrace instrument of Tracey Technologies, Corp., TX. The wave front analyzer 3 is connected electrically with the central processing unit 4, to which it delivers the pre-op data of the current patient and from which it gets commands to start communication with the multiplexer 5. Wi-fi modem 6 is controlled by the central processing unit 4 getting commands on uploading the patient's pre-op data from the wavefront analyzer 3 and other accompanying information, including patient's personal information from central processing unit 4 to the cloud library 1. Alternatively the predictive analytic tool is functional without the multiplexer and demultiplexer.

On the pre-op stage, based on the aberrometer collected data, the wave front analyzer provides the central processing unit with an index of the optical quality of the eye, an index of the optical quality of the cornea, an index of the lens optical dysfunction, and data on the tear film conditions. The central processing unit, upon getting the requested access to the MIMO cloud library, allows the wi-fi modem to transfer the data from the wave front analyzer to the multiplexer and to upload the data to the cloud library through one of its open multiple inputs. Through one of the open multiple-access cloud library outputs, the central processing unit gets the access to the data stored in the cloud library and chooses the pre-op data maximally correlated with the current patient's data. The requested data are downloaded to the central processing unit through the wi-fi modem and the demultiplexer. Operating interactively through the central processing unit, doctor adjusts the patient's conditions and the goals of the planned surgery to the post-op results, achieved with the surgery techniques, materials and instruments used by the surgeons of a certified level. After the surgery has been made, the post-op results accompanied by the surgery requisites are uploaded to the cloud library.

Particularly, the present invention provides a method that operates with digital twins, that is, with their modifiable parts. There are two stages: pre-operational and post-operational. Pre-operationally, the optometric characteristics of the total eye and of its components are measured, including refraction, light scatter, and eye structure, as well as their spatial distributions and time variations. From these characteristics, their digital twins are built, each one consisting of a modifiable part and of a non-modifiable part. A vector space is formed from the modifiable parts, each vector having an assigned correctable weight coefficient. To have criteria of quality, scoring metrics are built, that are a quality of vision index QVI, a cornea performance index CPI, and a dysfunction lens index DLI, each of them containing the related descriptors consisting of modifiable and non-modifiable parts of digital twins. Vector spaces of the current and of the previous cases are comparatively analyzed to define the best-fit vector space as a draft prediction for the upcoming medical procedure. From the best-fit vector space and non-modifiable part of the current-case digital twin, the scoring metrics are calculated. The draft-prediction vector space is modified by the correctable weight coefficients to maximize the related scoring metrics and to become a prediction score for the upcoming medical procedure. The predicted digital twin is reconstructed by combining the non-modifiable part of the pre-operational digital twin and the modified part of the digital twin derived from the best-fit vector space after its modification by the correctable weight coefficients. The pre-operational digital twin and the predicted digital twin are stored. Predicted data are displayed in a scoring metrics format and in a digital twin format.

Post-operationally, the following steps, made pre-operationally, are repeated. The optometric characteristics of the same patient, including their spatial distribution and temporal variations are measured. A digital twin based on optometric characteristics, measured post-operationally, is built. Vector space is formed from the modifiable part of the digital twin. Scoring metrics is created enabling to evaluate the refraction quality of the eye and of its components as their Refraction Quality Index (RQI) based on direct ray tracing measurements (“raw” data). Using polynomial approximation and calculated Modulation Transfer Function (MTF) along with the dynamics of tear film, the Cornea Performance Index is acquired. Adding the measured data on the light scatter in the eye, one can get the Quality of Vision Index (QVI). Subtracting the corneal component, the quality metrics of the crystalline lens known as Dysfunction Lens Index (DLI) is acquired.

Post-operational comparative analysis of the predicted and of the current post-operational vector spaces is made via machine-learning by correcting the weight coefficients aimed to maximize the scoring metrics, these correctives to be considered in the consecutive medical procedures. The results of post-operational analysis are displayed and stored. An artificial intelligence system may apply the machine-learning algorithm.

The proposed method can have other embodiments with modified parameters, or their specific range, or specific techniques of their acquisition. In one of these other embodiments, calculation of QVI considers the impact of higher order aberrations with the customized empirical correction.

In another embodiment, calculation of CPI considers the tear film dynamics based on the measured dynamics of the corneal topography and/or on the measured dynamics of the refraction of the total eye.

In yet another embodiment, calculation of DLI, when the higher-order aberrations of the internal optics are reconstructed by means of subtraction of the wave front, calculated for the cornea, from the wave front of the total eye, or by measuring the aberrations of the internal optics separately.

The indices QVI, CPI, DLI may have a decimal (0 to 10) scale, where the lowest quality is scored by 0, and the highest quality is scored by 10. Procedures of comparative analysis may be based on machine learning techniques using the k-nearest neighbours (kNN) or neural networking. The refractive part of the optometric characteristics can be measured by the ray tracing technology. The structural optometric characteristics may be measured by the technology of the optical coherence tomography. The wave front is described by polynomials or by splines.

A system, implementing the proposed method, contains a sensor subsystem, a decision-making subsystem, a storage subsystem, a control subsystem, and an intra-system data bus. The sensor subsystem consists of the devices measuring optometric characteristics including refraction, light scatter, and eye structure with their spatial distributions and variations in time. The decision-making subsystem consists of a twin builder, of a first metrics builder, of a second metrics builder, of a first builder of vector space, of a second builder of vector space, of a block of correction weight coefficients and of a processing unit. The storage subsystem contains preoperational memory, archived-cases memory and a dynamic interrogation switch. All cells of the archived-cases memory have connection with the decision-making subsystem via the dynamic interrogation switch. The control subsystem consists of a generator of control signals, of a display, and of an interface.

The system has the pre-operational and post-operational modes of operation with corresponding configurations. Switching between the modes is programmable. Each configuration is described separately for easier understanding.

In the pre-operational mode, the twin builder integrates the preoperational data from all sensors. It consists of two parts: modifiable and non-modifiable by a medical procedure. Both parts are delivered to the first metrics builder and to the storage subsystem. The first metrics builder generates the QVI, CPI and DLI metrics. The modifiable part of the digital twin is delivered to the first builder of vector space, which is connected to the first input of the processing unit, the second input of it being connected to the second builder of vector space. The second builder of vector space receives the digital twins from the archived-cases memory consecutively in time through the dynamic interrogation switch. The processing unit analyzes the vector spaces from the first and from the second builders of vector space generating an output for decision making. The processing unit delivers the modified part of the predicted digital twin to the second metrics builder and to the storage subsystem. The second metrics builder generates the QVI, DLI, and CPI scores of draft-predicted metrics that is further maximized using the block of correction weight coefficients.

In the post-operational mode, the decision-making subsystem has the same composition with some difference of functioning. The twin builder generates a post-operational digital twin, both parts of which are delivered to the first metrics builder and to the storage subsystem. The first metrics builder generates the post-operational quality of vision scores. The modifiable part of the digital twin is delivered to the first builder of vector space, that is connected to the first input of the processing unit, its second input being connected to the second builder of vector space. The second builder of vector space receives the digital twins from the archived-cases memory consecutively in time through the dynamic interrogation switch. The processing unit analyzes the vector spaces received from the first builder of vector space and from the second builder of vector space and to generates an output for decision making. The modified part of the post-op digital twin is delivered to the second metrics builder and to the storage subsystem. The non-modifiable part of the digital twin is delivered to the second metrics builder. The second metrics builder generates the post-operational scores of QVI, CPI and DLI integrating the data from the output of the processing unit. The processing unit maximizes these scores in the machine-learning way by correcting the weight coefficients and thus minimizing the difference between the predicted and the post-operational metrics. The outputs of the first and of the second metrics builders are displayed for possible machine-human interaction.

In a variation of the embodiments, the optometric devices are represented by a wave front sensing component and by an eye-structure measuring component. In another embodiment, the wave front sensing component is a ray tracing aberrometer. In yet another embodiment, the eye-structure measuring component is a corneal topographer and an optical coherence tomograph. In yet another embodiment, the wave front sensing component and the eye-structure measuring component are combined in a single design construction. In yet another embodiment, all sensors have integrated optics and mechanics with a common optical axis. In yet another embodiment, the ray tracing aberrometer is configured to measure intraocular distances, light scatter in the eye, tear film dynamics and the dysfunction of the crystalline lens. In yet another embodiment, the system is split in two parts (two and two, or three and one), one of them residing at the client site, another one resides in the cloud. This embodiment can have a multi-client configuration.

The method disclosed herein operates with digital twins, namely, with their modifiable parts. There are two stages: pre-operational and post-operational. Pre-operationally, the optometric characteristics of the total eye and of its components are measured, including refraction, light scatter, and eye structure, as well as their spatial distributions and time variations. From these characteristics, their digital twins are built, each one consisting of a modifiable part and of a non-modifiable part. A vector space is formed from the modifiable parts, each vector having an assigned correctable weight coefficient. To have criteria of quality, scoring metrics are built, that are: a quality of vision index QVI, a cornea performance index CPI, and a dysfunction lens index DLI, each of them containing the related descriptors consisting of modifiable and non-modifiable parts of digital twins. Vector spaces of the current and of the previous cases are comparatively analyzed to define the best-fit vector space as a draft prediction for the upcoming medical procedure. From the best-fit vector space and non-modifiable part of the current-case digital twin, the scoring metrics are calculated. The draft-prediction vector space is modified by the correctable weight coefficients to maximize the related scoring metrics and to become a prediction score for the upcoming medical procedure. The predicted digital twin is reconstructed by combining the non-modifiable part of the pre-operational digital twin and the modified part of the digital twin derived from the best-fit vector space after its modification by the correctable weight coefficients. The pre-operational digital twin and the predicted digital twin are stored. Predicted data are displayed in a scoring metrics format and in a digital twin format.

Post-operationally, the following steps, made pre-operationally, are repeated. The optometric characteristics of the same patient, including their spatial distribution and temporal variations are measured. A digital twin based on optometric characteristics, measured post-operationally, is built. Vector space is formed from the modifiable part of the digital twin. Scoring metrics is created enabling to evaluate the refraction quality of the eye and of its components as their Refraction Quality Index (RQI) based on direct ray tracing measurements (“raw” data). Using polynomial approximation and calculated Modulation Transfer Function (MTF) along with the dynamics of tear film, the Cornea Performance Index is acquired. Adding the measured data on the light scatter in the eye, one can get the Quality of Vision Index (QVI). Subtracting the corneal component, one can come to acquiring the quality metrics of the crystalline lens known as Dysfunction Lens Index (DLI).

Post-operational comparative analysis of the predicted and of the current post-operational vector spaces is made in a machine-learning way by correcting the weight coefficients aimed to maximize the scoring metrics, these correctives to be considered in the consecutive medical procedures. The results of post-operational analysis are displayed and stored.

Within the teachings of this invention, the proposed method may have other embodiments with modified parameters, or their specific range, or specific techniques of their acquisition. In one calculation of QVI considers the impact of higher order aberrations with the customized empirical correction.

In another embodiment, calculation of CPI considers the tear film dynamics based on the measured dynamics of the corneal topography and/or on the measured dynamics of the refraction of the total eye.

Yet another embodiment, considers calculation of DLI, when the higher-order aberrations of the internal optics are reconstructed by means of subtraction of the wave front, calculated for the cornea, from the wave front of the total eye, or by measuring the aberrations of the internal optics separately.

The indices QVI, CPI, DLI can have a decimal (0 to 10) scale, where the lowest quality is scored by 0, and the highest quality is scored by 10. Procedures of comparative analysis can be based on machine learning techniques using the k-nearest neighbours (kNN) or neural networking. The refractive part of the optometric characteristics can be measured by the ray tracing technology. The structural optometric characteristics can be measured by the technology of the optical coherence tomography. The wave front is described by polynomials or by splines.

A system, implementing the proposed method, contains a sensor subsystem, a decision-making subsystem, a storage subsystem, a control subsystem, and an intra-system data bus. The sensor subsystem consists of the devices measuring optometric characteristics including refraction, light scatter, and eye structure with their spatial distributions and variations in time. The decision-making subsystem consists of a twin builder, of a first metrics builder, of a second metrics builder, of a first builder of vector space, of a second builder of vector space, of a block of correction weight coefficients and of a processing unit. The storage subsystem contains preoperational memory, archived-cases memory and a dynamic interrogation switch. All cells of the archived-cases memory have connection with the decision-making subsystem via the dynamic interrogation switch. The control subsystem consists of a generator of control signals, of a display, and of an interface.

The system has the pre-operational and post-operational modes of operation with corresponding configurations. Switching between the modes is provided programmably. Each configuration is described separately for easier understanding.

In the pre-operational mode, the twin builder integrates the preoperational data from all sensors. It consists of two parts: modifiable and non-modifiable by a medical procedure. Both parts are delivered to the first metrics builder and to the storage subsystem. The first metrics builder generates the QVI, CPI and DLI metrics. The modifiable part of the digital twin is delivered to the first builder of vector space, which is connected to the first input of the processing unit, the second input of it being connected to the second builder of vector space. The second builder of vector space receive the digital twins from the archived-cases memory consecutively in time through the dynamic interrogation switch. The processing unit analyzes the vector spaces from the first and from the second builders of vector space generating an output for decision making. The processing unit delivers the modified part of the predicted digital twin to the second metrics builder and to the storage subsystem. The second metrics builder generates the QVI, DLI, and CPI scores of draft-predicted metrics that is further maximizanaled using the block of correction weight coefficients.

In the post-operational mode, the decision-making subsystem has the same composition with some difference of functioning. The twin builder generates a post-operational digital twin, both parts of which are delivered to the first metrics builder and to the storage subsystem. The first metrics builder generates the post-operational quality of vision scores. The modifiable part of the digital twin is delivered to the first builder of vector space, that is connected to the first input of the processing unit, its second input being connected to the second builder of vector space. The second builder of vector space receives the digital twins from the archived-cases memory consecutively in time through the dynamic interrogation switch. The processing unit analyzes the vector spaces received from the first builder of vector space and from the second builder of vector space and to generates an output for decision making. The modified part of the post-op digital twin is delivered to the second metrics builder and to the storage subsystem. The non-modifiable part of the digital twin is delivered to the second metrics builder. The second metrics builder generates the post-operational scores of QVI, CPI and DLI integrating the data from the output of the processing unit. The processing unit maximizes these scores in the machine-learning way by correcting the weight coefficients and thus minimizing the difference between the predicted and the post-operational metrics. The outputs of the first and of the second metrics builders are displayed for possible machine-human interaction.

In variation of embodiments, the optometric devices are represented by a wave front sensing component and by an eye-structure measuring component. In another embodiment, the wave front sensing component is a ray tracing aberrometer. In yet another embodiment, the eye-structure measuring component is a corneal topographer and an optical coherence tomograph. In yet another embodiment, the wave front sensing component and the eye-structure measuring component are combined in a single design construction. In yet another embodiment, all sensors have integrated optics and mechanics with a common optical axis. In yet another embodiment, the ray tracing aberrometer is configured to measure intraocular distances, light scatter in the eye, tear film dynamics and the dysfunction of the crystalline lens. In yet another embodiment, the system is split in two parts (two and two, or three and one), one of them residing at the client site, another one resides in the cloud. This embodiment may have a multi-client configuration.

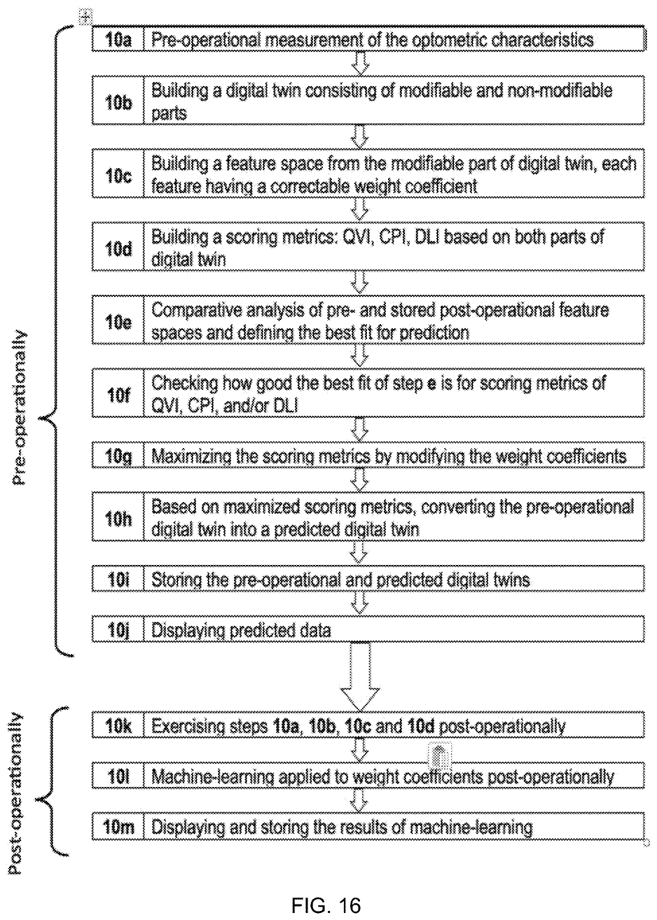

The method of predictive analytics of the quality of vision of a patient to be subjected to a medical procedure comprises the following steps (FIG. 16). First, the optometric characteristics of the total eye and of its components are measured (step 10a in FIG. 16) for a given patient, including their spatial distributions and time variations. These characteristics include refraction, light scatter, eye structure, that is its geometry, distances, tilts, etc. Acquisition of optometric characteristics are provided by the topographers, aberrometers, optical coherent tomographs, etc., separately or, in combination, all-in-one.

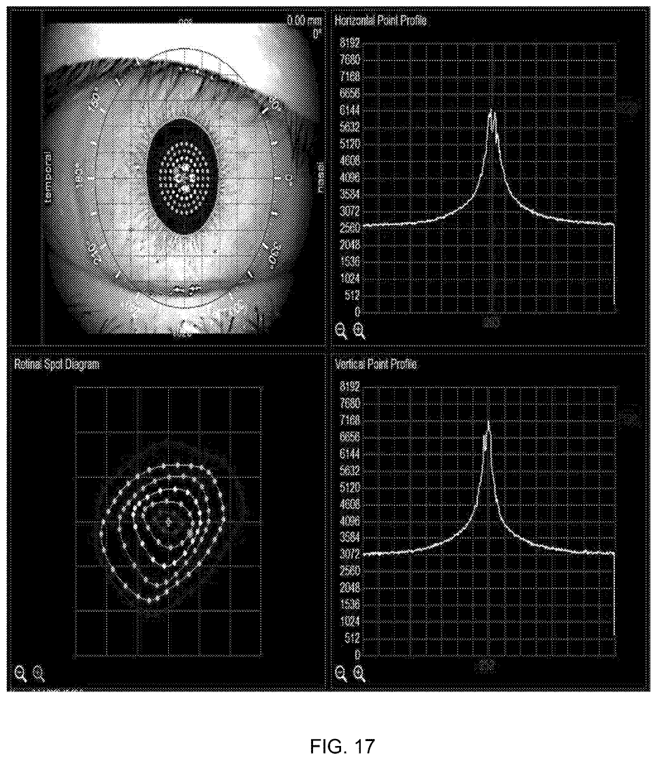

The proposed method operates with data acquired by these sensors. FIG. 17 illustrates the origins of initial ray tracing parameters. The left upper corner shows the coordinates of laser beam, hitting consecutively in time the aperture of the eye. The left lower corner shows the coordinates of laser beam arrival at the retina. The right part of the screen represents the shape of the signals received by the detectors, corresponding to the X-Y energy distribution in the laser projection on the retina for one of the entrance points.

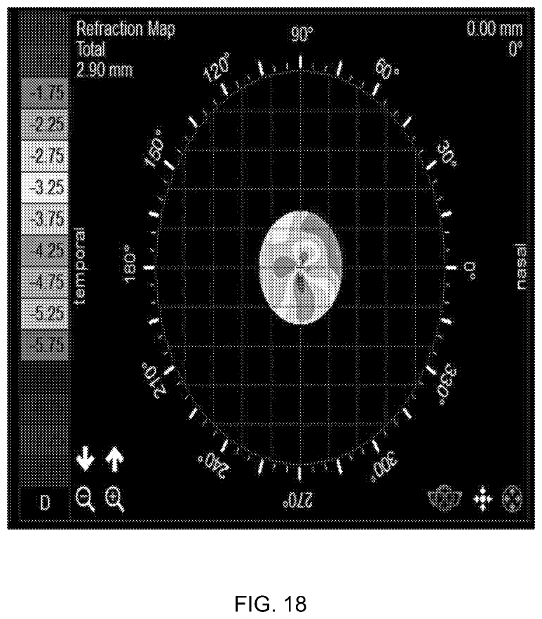

After processing the data for all entrance points, the distribution of refraction over the entrance aperture is got and displayed along with the values evaluating the state of refraction features of the eye optics (FIG. 18).

Illustration of the corneal part of the refraction features acquired with a topographer is presented in FIG. 19. The left upper side of the screen shows the projected circular structure of the light (Placido disks) reflected from the cornea. The corresponding distribution of refraction is shown below. To the right is the digitized description of the corneal topography in terms of Zernike polynomials and other generally used descriptors.

The metrics to be used in the prediction procedure can be calculated in two ways: (1) from “raw” data using the calculation of the degree of configuration change of a multidimensional feature space or, (2) from relations among predetermined polynomial components approximating the refraction features of the total eye and its cornea and lens.

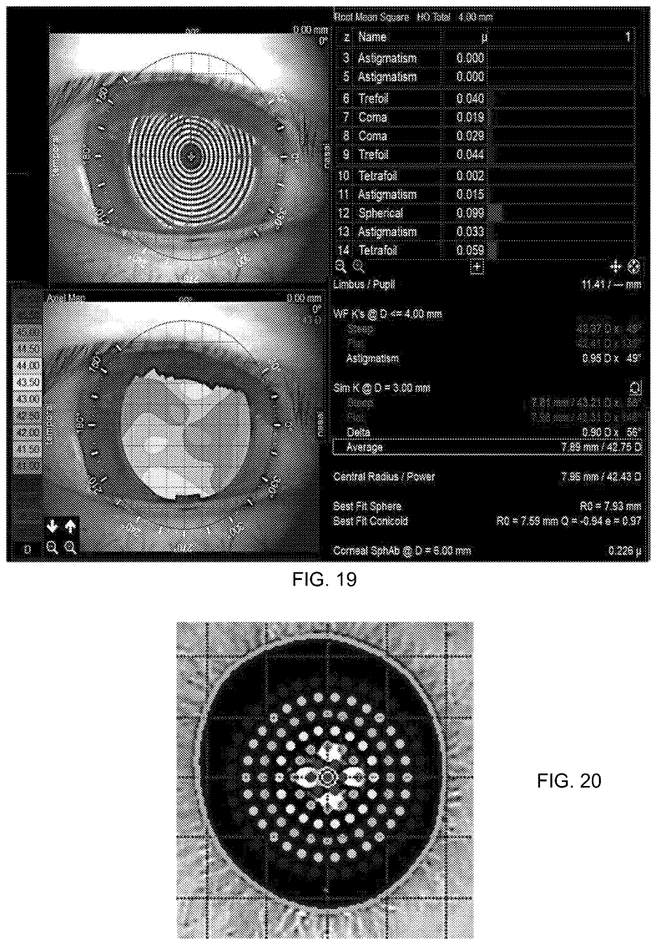

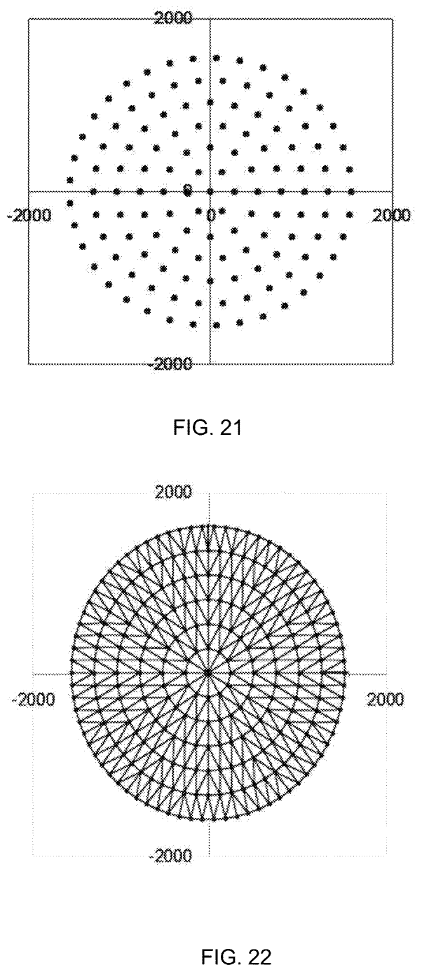

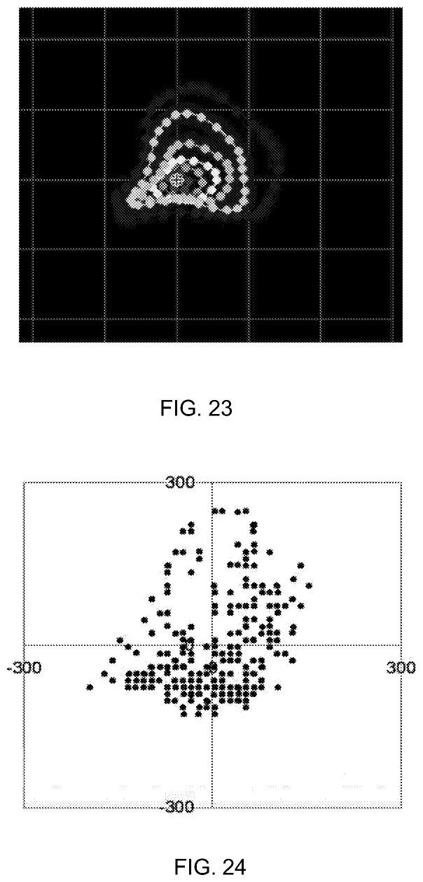

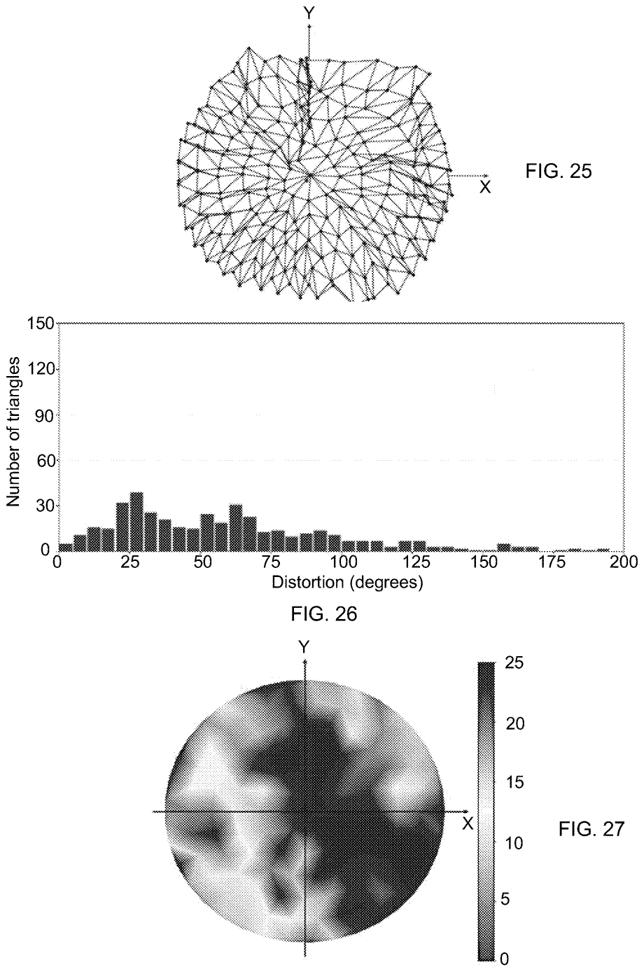

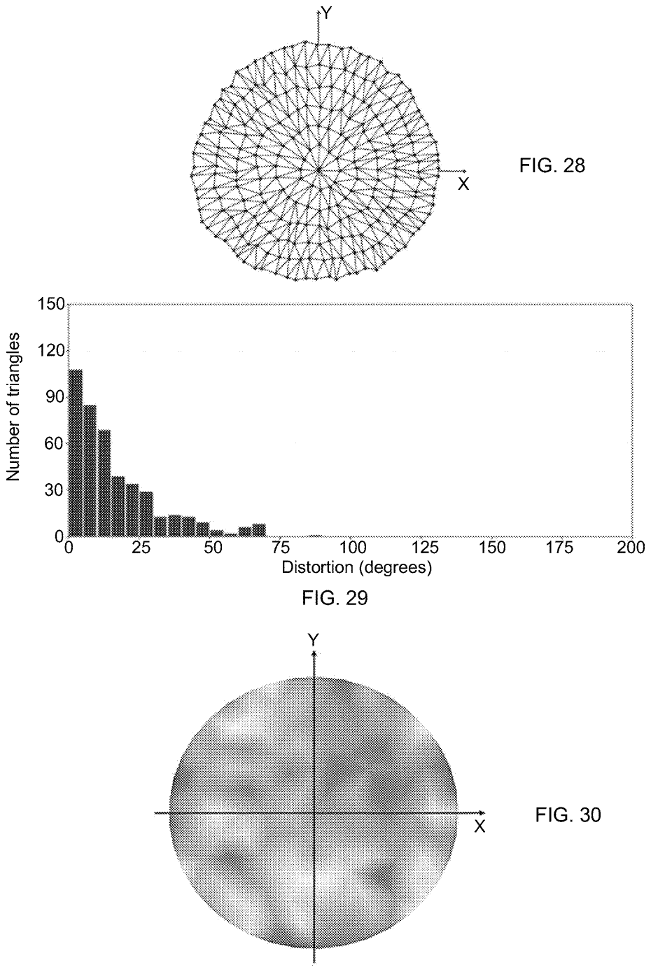

By following the first way, a reference space is defined as a two-dimensional X-Y distribution of eye probing coordinates (FIGS. 20-21). From this array of points, a reference triangular mesh is configured (FIG. 22). The comparison mesh is created by shifting the reference coordinates by the value of coordinates measured by the detectors (FIGS. 23-24). This new mesh (FIG. 25) is compared with the reference one of FIG. 22. For each triangle, the degree of distortion is measured, and corresponding histogram is reconstructed (FIG. 26). Refraction Quality Index (RQI) is defined as the ratio of the number of triangles within the visual acuity corresponding to a certain value, for example, 20/20, to the total number of triangles in the mesh. From the degree of similarity between corresponding pairs of these two meshes, a map of refraction quality is reconstructed (FIG. 27). Within a decimal scale, the RQI=1.9 for the case of FIGS. 25-27 measured pre-operationally. The same eye, examined post-operationally showed QRI=8.9 (FIGS. 28-30).

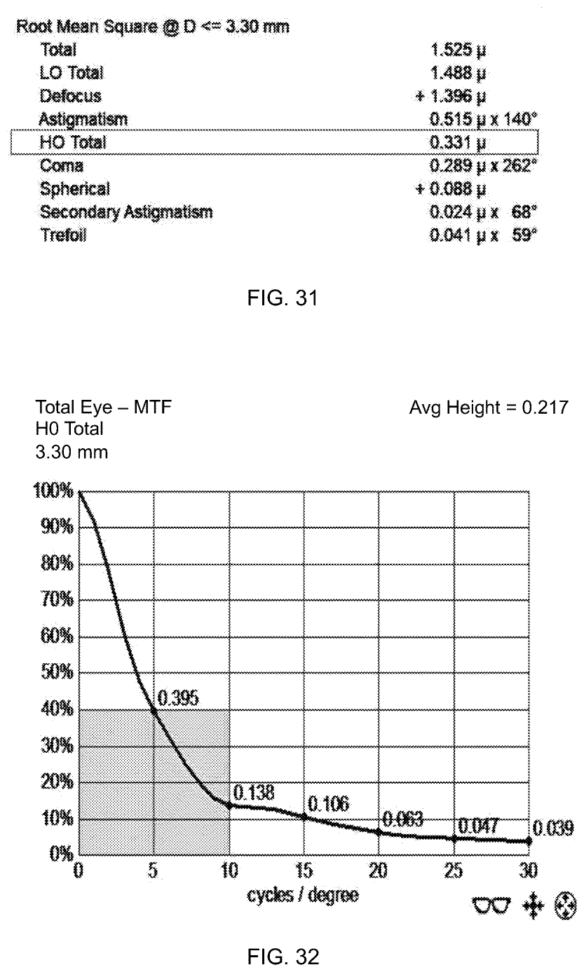

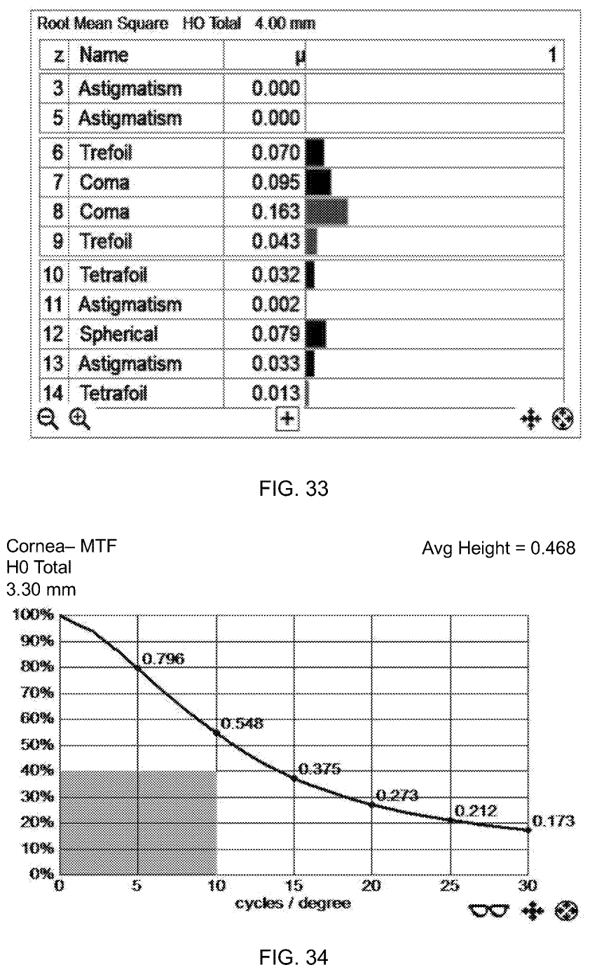

Following the second way, a polynomial description is acquired (FIG. 31) for reconstruction of the wavefront and for the refraction distribution of the total eye. Modulation Transfer Function (MTF) represented in FIG. 32, along with the selected combination of polynomials, serves for calculation of the Quality of Vision Index (QVI). Similarly, from the polynomials (FIG. 33) and MTF (FIG. 34), the CPI is calculated. Subtracting the corneal wavefront from the wavefront of the total eye, similar mathematical transforms result in reconstruction of the Dysfunction Lens Index (DLI).

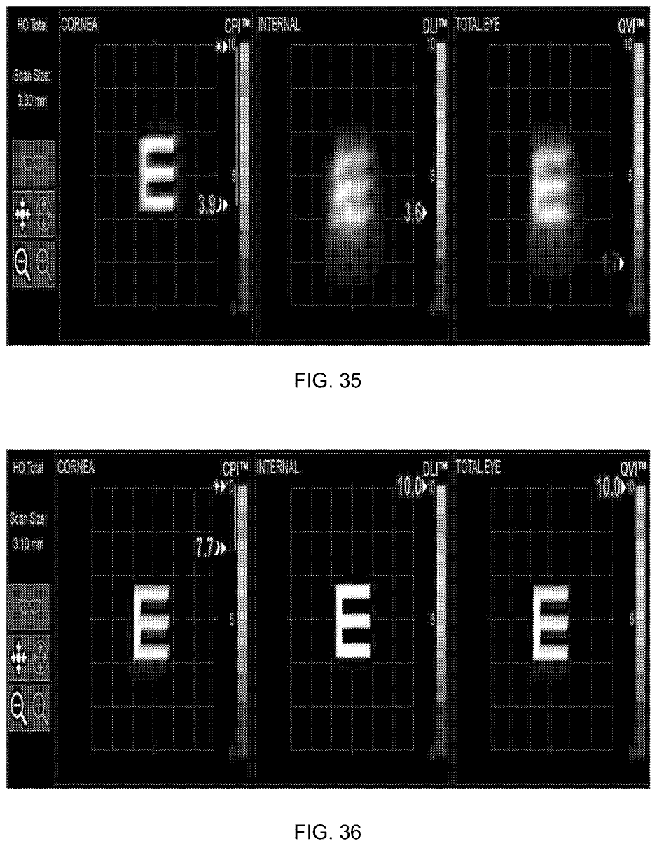

FIGS. 35-36 are the screenshots from the ITRACE aberrometer/topographer (Tracey Technologies, Corp.) demonstrating the metrics for two different patients having different proportions between indices in the eye with bad vision QVI=1.7 (FIG. 35) and in the eye with good vision QVI=10.0 (FIG. 36).

Of these characteristics, their digital twin is built, i. e., physical descriptions are converted into properly organized machine language codes (step 10b). The constructed digital twin consists of two parts, one of them being modifiable in the course of the medical procedure, another one being non-modifiable during the medical procedure.

The modifiable part of the digital twin represents an operational space consisting of twin modifiable fragments for each of the optometric characteristics, each fragment being a multidimensional entity, or, in mathematical terms, a multidimensional feature, or, in more abstract language, a multidimensional vector. Building the operational vector space is represented in FIG. 16 as step 10c. Each vector (entity, feature) may be modified by changing its weight coefficient.

To provide human-machine interaction requiring the human perceivable information, a scoring metrics of the quality of vision is built (step d) being represented by a refraction quality index (RQI), a quality of vision index QVI, by a cornea performance index CPI, and by a dysfunction lens index DLI. Each of these indices is created by a mathematical transform of related optometric characteristics, the mathematical transform including the empirically confirmed correction. Similarly, to digital twins, the scoring indices originate from both modifiable and non-modifiable fractions. It should be mentioned that not only aberrations are the data for indices calculation. Light scatter, tear film stability are the data components included in the calculation.

Calculation of the dysfunction lens index DLI that scores the quality of the internal optics of the eye (practically, of the crystalline lens) encountering the contribution of higher order aberrations measured for the internal optics either by means of subtraction of the wave front calculated for the cornea from the wave front of the total eye, or by measuring separately the fraction of the aberrations produced by the internal optics.

The disfunction lens index DLI and the quality of vision index QVI (scoring the quality of vision of the total eye) are quantified not only by the amount of higher order aberrations, but also by their types (V. Molebny, S. Molebny. “Ocular Q-factor: an approach to eye aberrations analysis”, Journal of Modern Optics, 2011, Vol. 58, No. 19-20, pp. 1729-1735). When constructing the math for calculation of the QVI, empirical adjustment is usually incorporated in the proprietor software of the aberrometer, QVI being displayed in a certain range for convenience of analysis by doctors (Zh. Li, et al. “Dysfunctional lens index serves as a novel surgery decision-maker for age-related nuclear cataracts”, Current Eye Research, Taylor&Francis online journal, Mar. 1, 2019). Such presentation of scoring makes the DLI easier to be used as a cut-off value for surgery criterion.

Calculation of the cornea index CPI scoring the cornea's fraction in the quality of vision includes not only the data from the topographer, but also the conditions of the tear film dynamics. All above-mentioned indices, the quality of vision index QVI, the cornea performance index CPI, and the dysfunction lens index DLI have a decimal 0 to 10 scale, where the lowest quality is scored by 0, and the highest quality is scored by 10.

The next step (step 10e) consists in the comparative analysis of the pre-operational vector space with the post-operational vector spaces acquired in the medical procedures of the same kind from other patients, being stored in the database. This comparative analysis results in defining the best fit vector space for the upcoming medical procedure. Based on these results, calculation of the scoring metrics of the quality of vision is made (step 10f). As an example, for the cataract surgery, DLI and QVI are determined. These indices reconstructed from the best-fit vector space are modified to reach their maxima (step 10g). Modification of the best fit vector space is made automatically according to the program. As an option, a human operator can be included in the process to make any correction manually.

To operate the machine learning procedures, after the best fit vector space has been corrected either by a human operator, or by a program, transformation of the pre-operational digital twin into a predicted digital twin is performed (step 10h) based on the combination of the non-modifiable part of the pre-operational digital twin and of the modified part of the digital twin derived from the best fit vector space after its modification by the correctable weight coefficients.

Both digital twins, the pre-operational one and the predicted one are stored for further processing (step 10i). Predicted data in a scoring metrics format and in a digital twin format are displayed (step 10j), thus becoming perceivable for the human participation in the process of machine learning, would it be expedient.

The next step aimed to encounter the acquired experience (step 10k) starts after the medical procedure has been performed. It repeats steps 10a, 10b, 10c and 10d. A post-operational vector space is created from the actual post-operational optometric characteristics acquired and processed in the same way as for building the pre-operational vector space.

In step 10l, a comparative analysis of the predicted and of the post-operational vector spaces is performed in a machine-learning procedure that incorporates the experience of maximization of the quality of vision indices, provided in steps 10g and 10h. The results of machine learning are displayed and stored (step 10m).

One of the options of machine learning is based on the k-nearest neighbours (kNN) algorithm that operates on the search of similar data points in the nearest neighbourhood of’ one another in the vector space. Any vector can be varied and controlled by its weight coefficient. The entire training dataset is stored, and the choice is performed at the time of prediction of the outcome of the medical procedure, and repeated post-operationally as a next iteration of learning.

In another option of machine learning, the layers of neural networks are created for all the features of each stored digital twin. Trial and error determination process is provided by varying the weight coefficients and verifying the scoring indices values to be maximized. The training stage is provided each time after a new case is stored.

Optometric characteristics can be measured with any type of instrumentation. As an example, aberrations can be measured based either on ingoing (ray tracing) or outgoing (Shack-Hartmann) principles. The intraocular distances can be measured using optical coherence tomography, or triangulation principle. The last one can be combined with ray tracing. That is why, the ray tracing may be preferable since both kinds of data can be acquired in a single instrument.

For the description of aberrations, wave front reconstruction may be based either on approximation (polynomial description) or on interpolation technique (splines). The choice depends on the alternative: smoothed, filtered data, or not to-be-lost small details.

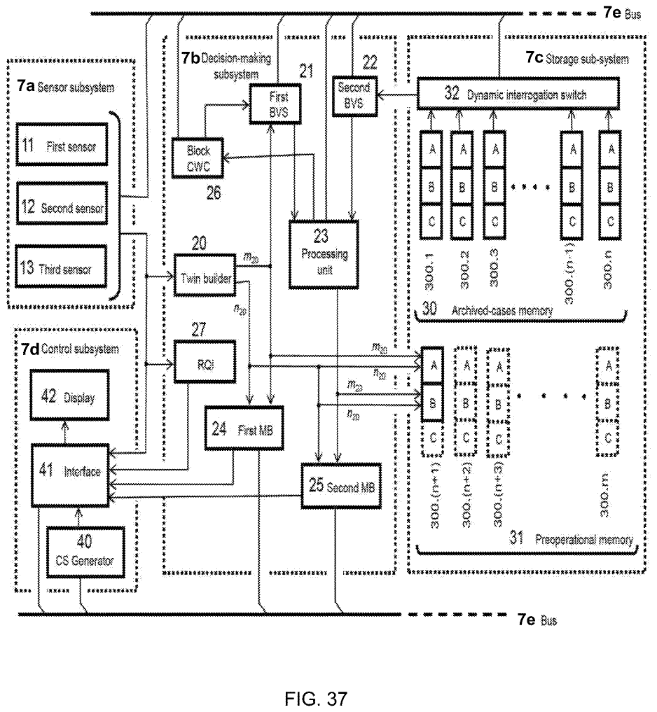

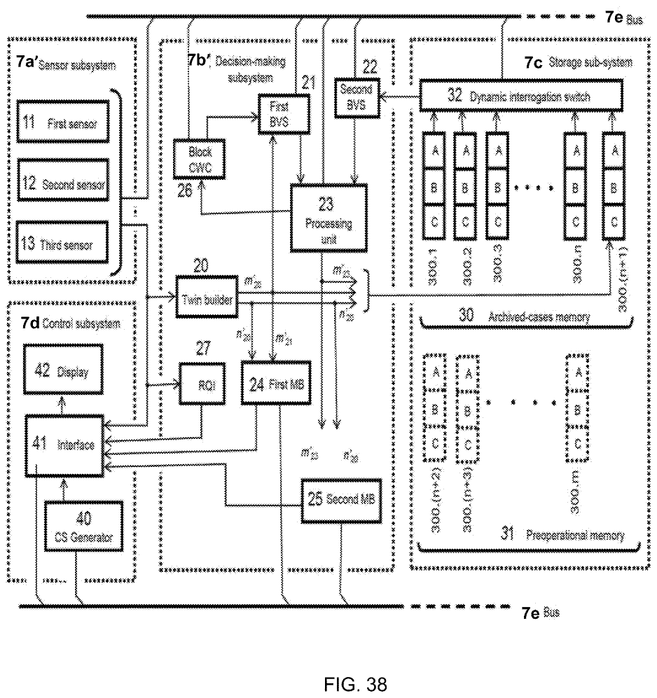

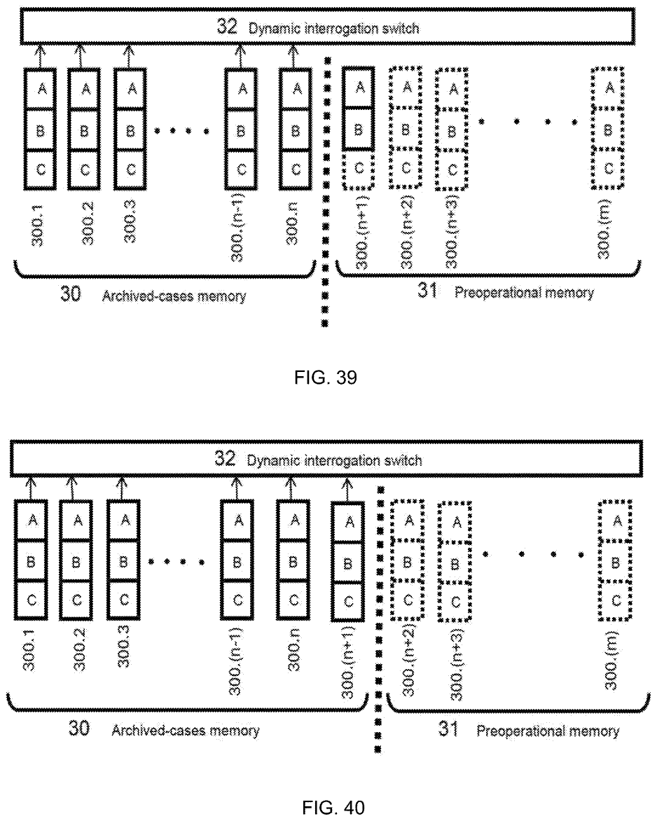

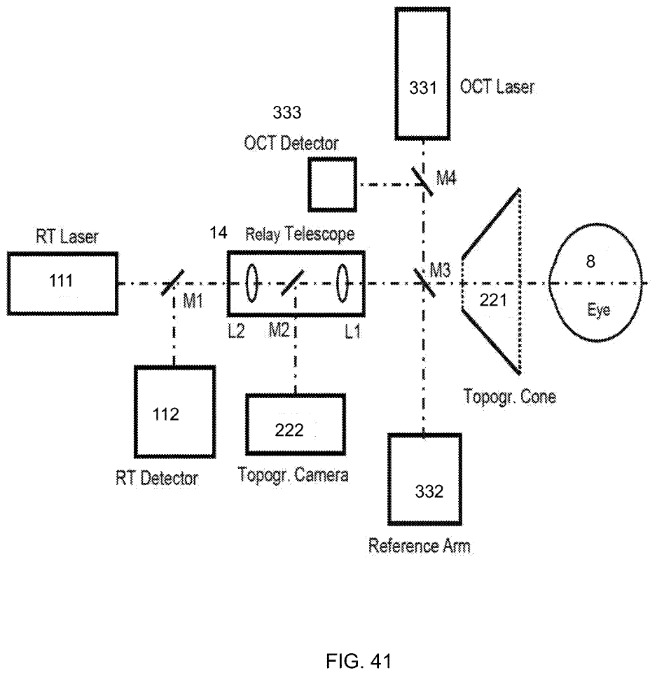

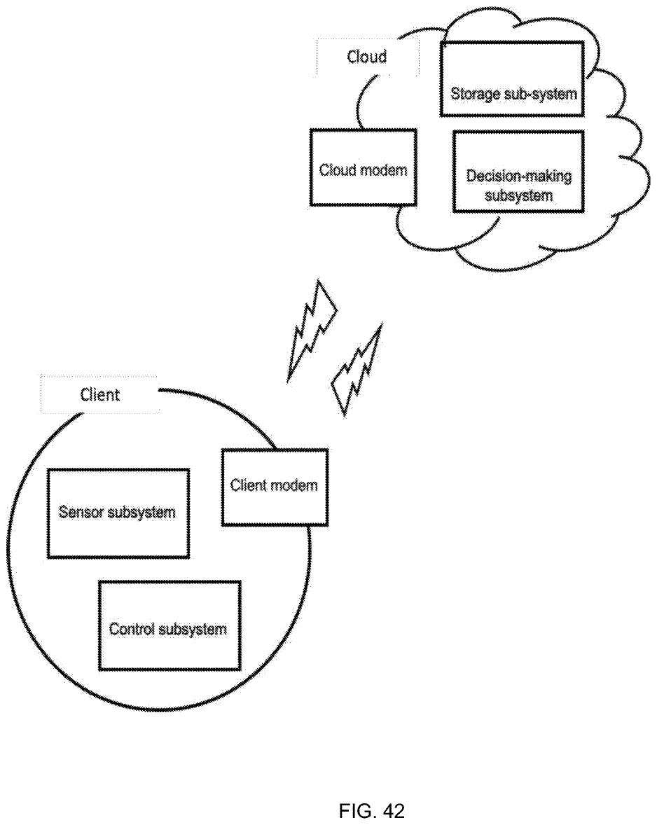

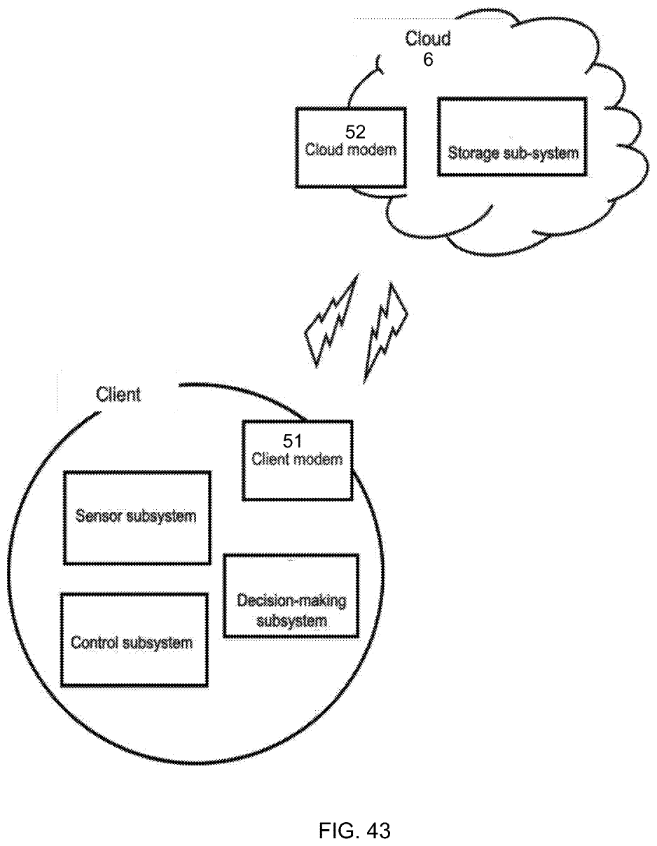

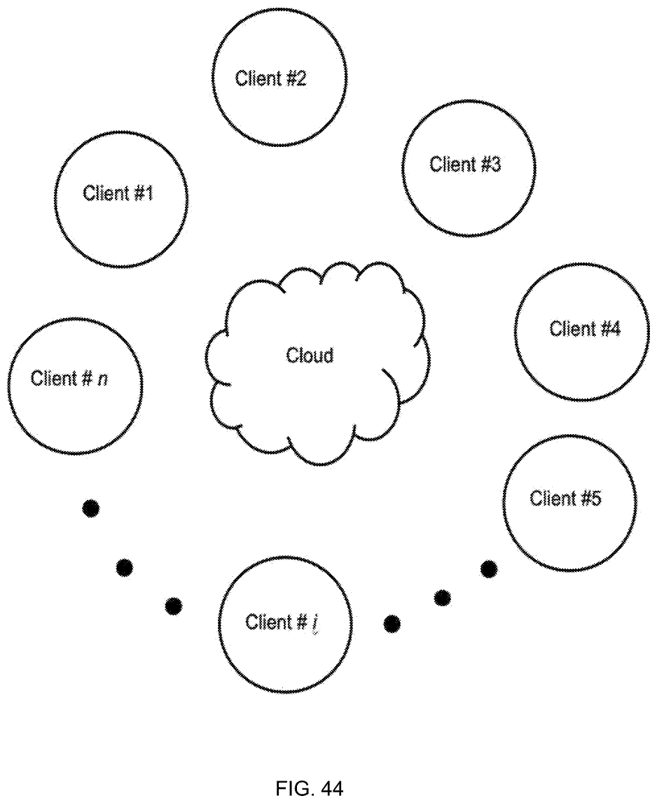

The described method of predictive analytics is materialized in a system whose functional schematic in the pre-operational configuration is given in FIG. 37. The system 7 has a sensor sub-system 7a, a decision-making sub-system 7b, a storage sub-system 7c, control subsystem 7d, and an intra-system data bus 7e.

Sensor subsystem 7a contains optometric devices measuring the optometric characteristics of the total eye and of its components. These devices measure refraction of the total eye, the topography of the cornea, light scatter in the lens and in the cornea, eye structure that includes the intraocular distances. For these functions, the sub-system 7a contains a first sensor 11, a second sensor 12, and a third sensor 13. There could be other sensors, their number and delivered information are not limited by this invention. As an example, the sensors 11, 12 and 13 may be: an aberrometer 11, a topographer 12, an optical coherence tomograph 13. For aberrations measurement, as an aberrometer 11, any of the market-available devices for wave front measurement may be used: based on laser ray tracing principle, on Hatmann-Shack approach, on Tscherning approach, etc. The topographer 12 may use projection of structural light, like Placido disk, checkerboard or point-like structure. The role of the optical coherence tomograph 13 is to provide information on the structure of the eye, especially on the thickness of the cornea and on the intra-ocular distances defining the position and orientation of the crystalline lens.

The decision-making subsystem 7b consists of a twin builder 20, of a first builder of vector space 21, of a second builder of vector space 22, of a processing unit 23, of a first metrics builder 24, of a second metrics builder 25, of a block of weight coefficients 26, and of a block 27 for calculation of the Refraction quality index (RQI).

The storage subsystem 7c consists of a block of archived-cases memory 30, of a block of preoperational memory 31, and of a dynamic interrogation switch 32. The block of archived-cases memory 30 and the block of preoperational memory 31 have m per-patient cells in total, n of them being in the archived-cases memory, and (m-n) are reserved for the preop-cases memory. In the block of archived-cases memory 30, the cells are numbered as 300.1, 300.2, 300.n. In the block of preop-cases memory 31, the per-patient cells are numbered as 300.(n+1), 300.(n+2), . . . , 300.m. Each of the per-patient cells 300.i has three sub-cells: A, B and C. Sub-cell A contains, or is designated to contain, information on a pre-operational twin of a certain patient, a sub-cell B contains, or is designated to contain, information on a predicted twin of the same patient. A sub-cell C contains, or is designated to contain, information on a post-operational twin still of the same patient. Sub-cells filled with information are framed with solid lines, sub-cells reserved for filling are framed with dotted lines.

The control subsystem 7d contains a control-signals generator 40, an interface 41 and a display 42. The control subsystem 4 talks with the subsystems 7a, 7b and 7c via the intra-system data bus 7e and the direct connections using the control-signals generator 40 and the interface 41. It allows also the human operator to interact with the system perceiving the information via the display 42. The interface 41 has its connections with the outputs of the sensor subsystem 7a and the outputs of the first metrics block 24 and of the second metrics block 25 directly. Talking with the first block of vector space 21 and the second block of vector space 22 is provided through the intra-system data bus 7e.

Step 10a of the method functioning considers measuring pre-operationally the optometric characteristics of the total eye and of its components, their spatial distributions and time variations, including but not limited to refraction, light scatter, and eye structure. As mentioned above, these functions are exercised by the sensors 11, 12 and 13. Their output information is shared by the twin builder 20 and the display 40.

Step 10b considers building pre-operationally a digital twin based on measured optometric characteristics, the digital twin consisting of a modifiable part and of a non-modifiable part. The digital twin is created in the twin builder 20 having two outputs: output m delivering the modifiable part of the twin and output n delivering the non-modifiable part of the twin. For convenience, these outputs can be mentioned as m20 and n20. The output m20 is delivered to the first builder of the vector space 21, to the first metrics builder 24, and to the sub-cell A of the cell 300.(n+1) of the preoperational memory 31. The output n20 is delivered to the first metrics builder 24, to the second metrics builder 25, and to the sub-cell A of the cell 300.(n+1) of the preoperational memory 31.

Step 10c considers building pre-operationally a vector space from the modifiable part of the pre-operational digital twin providing each vector with a correctable weight coefficient. The pre-operational vector space is configured by the first block of vector space 21, connected to the block of correcting weight coefficients 26. That is controlled by the processing unit 23.

Step 10d considers building pre-operationally a scoring metrics of the quality of vision representing it by a quality of vision index QVI, by a cornea performance index CPI, and by a dysfunction lens index DLI, each of them containing the related descriptors consisting of modifiable and non-modifiable parts of the pre-operational digital twin. The creation of any of these indices is provided by the first metrics block 24 with its inputs directly from the twin builder 20. As mentioned before, examples of displayed indices for two different eyes are given in FIGS. 35-36. Refraction quality index (QRI) is calculated and displayed each time in the course of the exam

Step 10e considers comparative analysis of the pre-operational vector space with the post-operational vector spaces acquired in the same medical procedures of other patients, being stored in the database. The comparative analysis results in defining the best fit vector space for the upcoming medical procedure. This best fit is accompanied by calculation of the scoring metrics of the quality of vision based on measured and predicted data (step 10f), the scoring metrics being represented by a quality of vision index QVI, a cornea performance index CPI, and a dysfunction lens index DLI. The best fit can further be corrected by the correctable weight coefficients to maximize the related scoring metrics calculated in this step and presenting it as a predicted score for the upcoming medical procedure (step 10g).

The comparison is provided by the processing unit 23, having the input of the pre-operational vector space from the first block of vector space 21 and the input of the post-operational vector spaces from the second block of vector space 22. The latter one receives the data from the archived-cases memory 30 via the dynamic interrogation switch 32. The cells from 300.1 to 300.n are queried by the signals from the control-signals generator 40. Responding to the queries, each cell provides three vector spaces: pre-operational, predicted and post-operational back-reconstructed from the digital twins stored in the corresponding sub-cells A, B and C. The best fit vector space determined by the processing unit 23 is delivered to the second metrics builder 25 and to the sub-cell B of the cell 300.(n+1) of the pre-operational memory 31 as the modifiable part of the digital twin, whose non-modifiable part is delivered from the non-modifiable output n20 of the twin builder 20. The pre-operationally built metrics from the first metrics builder 24 and the predicted metrics from the second metrics builder 25 are delivered to the display 42 via the interface 41 following the commands from the control-signals generator 40.

The described interaction provides reconstruction of the pre-operational digital twin into a predicted digital twin (step 10h), storing the twins (step 10i) and displaying the predicted data (step 10j).

With step 10j, the prediction mode of system functioning comes to its end. But for the future cycles of prediction, it is expedient not to lose the experience of the last cycle. Therefore, the patient is examined post-operationally after the recovery period. This examination starts the machine learning mode of system functioning. With its start, cell 300.(n+1) is to be moved from the pre-operational memory 31 to the archived-cases memory 30. For programming, it means the change of its address.

The post-operational functioning of the proposed system in the machine learning mode can be better explained by FIG. 25. The illustrated functional diagram of the system contains the same subsystems with the same components, but with the modified configuration aiming to consider the experience acquired in the executed medical procedure and to make the prediction more accurate. The system itself is the same, its internal connections are switched to be adapted for the post-operational analysis. The machine learning mode starts with the steps analogous to pre-operational steps 10a, 10b, 10c, and 10d. They are brought together as a stage k in the post-operational functioning, consisting of steps ka, kb, kc, and kd.

Step ka considers measuring post-operationally the optometric characteristics of the same patient, including their spatial distribution and temporal variations. For this purpose, the same sensors are used: the first sensor 11 that could be an aberrometer of any kind and technology (recommended to be the same as in step 10a); the same second sensor 12, for example, a topographer; the same third sensor 13 (optical coherence tomograph).

Step kb considers building post-operationally a digital twin based on optometric characteristics measured post-operationally in step ka. The input of twin builder 20 is connected to the outputs of sensors 11, 12, 13. The digital twin, created by the twin builder 20, consists of modifiable (m) and non-modifiable (n) parts at the outputs m20 and n20 correspondingly. After creation of the post-operational twin, it is stored in the sub-cell C of the cell 300.(n+1) that changed its address to the archived-cases memory 30. The transition of the patient-personalized cell 300.(n+1) from the pre-operational memory 31 to the archived-cases memory 30 is illustrated by FIGS. 23-24. Until the sub-cell C is vacant, the cell is marked as pre-operational. All cells of the pre-operational memory are vacant until the next cycle of prediction for a new case/patient.

Step kc considers building a post-operational vector space from the modifiable part of the digital twin created post-operationally in step kb. Post-operational vector space is created by the first builder of vector space 21. Each vector has a correctable weight coefficient that can be changed by the block of correctable weight coefficients 26. Twin builder 20 sends the post-operational digital twin to the sub-cell C of cell 300.(n+1) that is moved by this action from the pre-operational memory 31 to the archived-cases memory 30.

Step kd considers building the post-operational scoring metrics of the quality of vision as represent by the post-operational quality of vision index QVI, by the post-operational cornea performance index CPI, and by the post-operational dysfunction lens index DLI. The outputs of twin builder 20 are connected to the inputs of the first metrics builder 24 that implements the step kd.

Step 20l considers post-operational comparative analysis of the predicted and of the current post-operational vector spaces in a machine-learning way by correcting the weight coefficients to achieve the maximal value of the scoring metrics. Weight coefficients are generated in the block of correctable weight coefficients 26 controlled by the processing unit 23. The first builder of vector space 21, manipulating with the generated weight coefficients and the modifiable part m20 from the twin builder 20, generates a new vector space, modifiable part of which is sent to the processing unit 23. Another input of the processing unit 23 receives the modifiable parts of digital twins from the archived cases memory 30 via the second builder of vector space 22. Modifiable output m23 and non-modifiable output n20 create a virtual digital twin, from which a virtual metrics is created in the second metrics builder 25. The process is repeated until the maximal value of virtual metrics is achieved.

In step 10m, the set of correcting weight coefficients is stored in archived cases memory 30 in separate cells as additional adjustment information for the future cases. In FIG. 25, it is addressed to the sub-cell C of cell 300.(n+1). The metrics from the second metrics builder 25 is displayed on the display 42.

The system embodiments can have modifications. In one of them, the sensor sub-system comprises a wave front aberrometer as the first sensor 11, a corneal topographer as the second sensor 12, and an optical coherence tomograph as the third sensor 13. In their combination, they provide the information pre-operationally and operationally on the optometric characteristics of the total eye (the wave front aberrometer and optical coherence tomograph) and of its components (with the corneal topographer inclusive), that are: refraction and all its derivatives, light scatter, eye structure, their spatial distributions and time variations.

As a modification of the main embodiment, the wave front aberrometer (being the sensor 11), if based on the ray tracing principle, can measure, additionally to the above mentioned, the following optometric characteristics: the intraocular distances (as described in PCT Publication WO2024/118883, 2024, V. Molebny, et al. “Method and device for laser ray tracing measurement of intraocular distances and structures:), the tear film dynamics (as described in Publication WO2025/212890, 2025, V. Molebny, et al. “Method and device for analysis of the ocular tear film dynamics,), the optical dysfunction of the crystalline lens (as described in Publication WO2024/228982, 2024, V. Molebny, et al. “Method and device for measuring optical quality of the human eye and its cristalline lens”,).

Still another embodiment consists in transforming the combination of sensor 11, sensor 12 and sensor 13 in the optically and mechanically integrated system with the principal feature of having a common optical axis for all three sensors. In a simplified schematic, showing only the main optical units and their optical axes, FIG. 26 illustrates such an embodiment, where the wave front aberrometer is represented by a unit 111 that is a laser for ray tracing and a unit 112 that is a two-dimensional position sensing detector. Detailed description of ray tracing aberrometry is found elsewhere (for example, in the “Handbook of Visual Optics”, P. Artal, Ed., CRC Press, 2017, Vol. 2, pp. 17-36, “Wavefront sensors” by V. Molebny).

Corneal topographer is represented by a unit 221 that is a topographer cone and a unit 222 that is a video camera for imaging the reflected regular structure, for example, concentric structure of the Placido disk (see, for example, “Corneal Topography in the Wavefront Era”, M. Wang, Ed., Slack Inc., 2006, pp. 177-188, “The iTrace combination corneal topography and wavefront system by Tracey Technologies” by J. Wakil et al.).

The optical coherence tomograph (OCT) is represented by a unit 331, that is an OCT laser source, a unit 332, that is a reference arm equalizing the path to the measured structures of the eye 8, and a unit 333, that is an OCT detector receiving the interfering signals, reflected from the eye structures and from the reference arm. The type of the OCT depends on the specification of the total system and can be better understood from the professional publications, for example, from the book “Optical Coherence Tomography” W. Drexler and J. Fujimoto, Eds., Springer, 2nd Edition, 2015, Pt. 2 “OCT Technology”, pp. 141-788.

A relay telescope 14 assists the optics of the unit 112 to conjugate the planes of the retina and of the position sensing detector of the unit 112. Mirrors M1, M2, M3, M4 have a functions of sensor integration and optical paths partitioning. The mirrors M1 and M4 are single-wavelength beam splitters: M1—the wave length of the wavefront sensor, M4—the wave length of the OCT, M2—transmitting the wave length of the wavefront sensor and reflecting the visible spectrum of the topographer, M3—transmitting the wave lengths of the topographer (visible) and of the wavefront sensor (near-to-visible infrared), and having the reflectance/transmittance ratio higher than 1:1 for the OCT wave length that is longer than the wave length of the ray tracing laser 111.

In yet another embodiment, the system is split into two parts (FIG. 27). The first one is a combination of three subsystems: the sensor subsystem, the decision-making subsystem and the control subsystem. The exchange of signals and data between this three-subsystem part and the storage subsystem is provided through electric or wireless communication line. In the wireless version, the storage subsystem 7c resides in a cloud. The communication line has a client modem 51 and a cloud modem 52. This embodiment requires intensive loading of the wireless communication line when analyzing data from the archived-cases memory 30.

An embodiment with another way of splitting the subsystems can resolve the problem of heavy traffic between the decision-making subsystem and the storage subsystem by combining them in a separate part. To implement this solution, the twin builder should be moved from the decision-making subsystem to the sensor subsystem. In this reconfiguration, the sensor subsystem is denoted as 7a′, and the decision-making subsystem is denoted as 7b′. With these changes, a two-part combination (7a′ & 7d} resides at a client site, and a two-part combination {7b′ & 7c} resides at the cloud site (FIG. 28).

Still another embodiment of the system (FIG. 29) comprises multiple two-part combinations {7a′ & 7d} residing at corresponding client sites (Client 1, Client 2, . . . Client n). A two-part combination {7b′ & 7c}, residing at the cloud site, is a common one that provides service for all clients via the internet connection.

Other and further aspects, features, benefits, and advantages of the present invention will be apparent from the following description of the presently preferred embodiments of the invention given for the purpose of disclosure.

### EXAMPLE 1

### Dysfunctional Lens Index (DLI)

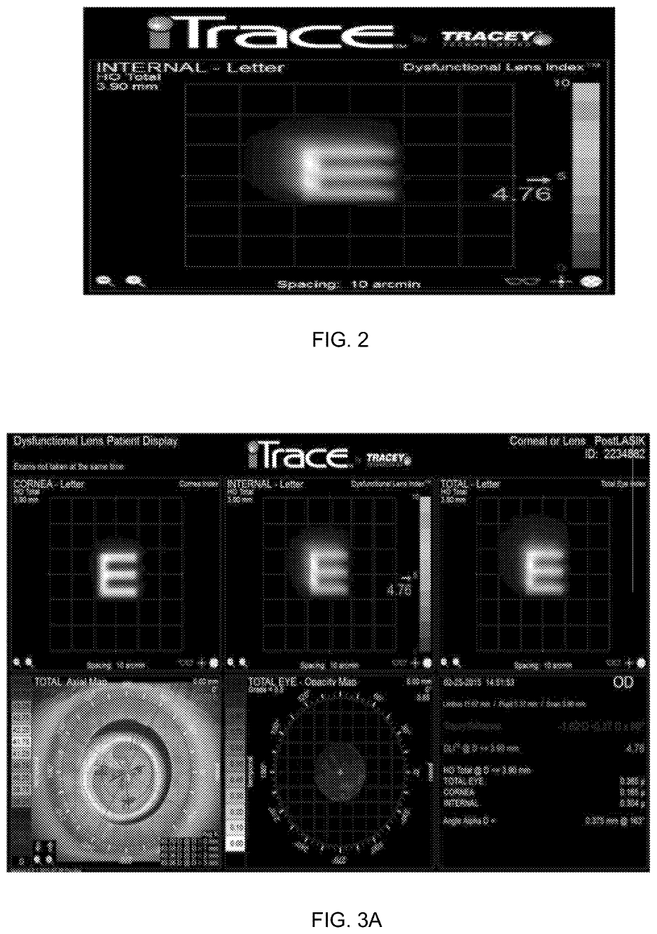

The Dysfunctional Lens Index (DLI) is a singular number that is calculated from the internal optics of the eye with the vast majority contributor being the lens (natural human crystalline lens or a man-made intraocular lens). The DLI is calculated from higher order aberrations within the internal optics of the eye as calculated by an aberrometer, most preferably through a true forward Ray Tracing aberrometer device, but can similarly be generated through other similar wavefront type devices such as those using a Hartmann-Shack sensor. The DLI scores the quality of vision from the Internal Optics (being predominately the lens) on a 0 to 10 scale. A lower number correlates to a more dysfunctional lens that negatively effects the quality of vision for the patient, which can indicate an early cataract. Essentially a low DLI correlates with an increase in the magnitude of higher order aberrations and predominantly is associated with early cataract formation (especially when below 5.0—see reference) in those patients over 50 years of age. Evaluating the DLI after cataract surgery is also a useful tool in evaluating the quality of vision and performance of man-made intraocular lenses as if DLI is high and close to 10 then that indicates very good optical lens performance with a DLI score of q0 being ideal promoting excellent vision. It is important to note that the DLI along with all the Quality of Vision indices do not count on the measurement of refractive error from sphere and cylinder. FIG. 2 shows a DLI scale where green on the color scale is “good” and warmer colors equate to reduced visual function. The letter “E” image reflects quality of vision from internal optics (Lens).

### Dysfunctional Lens Syndrome

Stage I of the aging lens occurs from the mid 40s to early 50s with a loss of accommodation and lens aberrations. In this stage the lens hardens and loses accommodation, i.e., presbyopia, lens high order aberrations develop with DLI<5 and quality of vision decreases. Stage II of the aging lens occurs from the mid 50s to the 60s and includes a forward lens scatter of light, in addition to stage I symptoms. In this stage there are increased lens higher order aberrations where DLI<4, small Lens protein aggregation causes forward light scatter, i.e., glare, and the patient sees bright colors as more “Brown”. Stage III of the aging lens occurs from the 60s to the 80s and includes a backscatter of light and lens opacity (cataract) in addition to stages I and II symptoms. In this stage there is a large lens protein aggregation which causes a backscatter of Light or opacity and a vision function decline with poor contrast sensitivity, and glasses are unable to correct to any normal acuity.

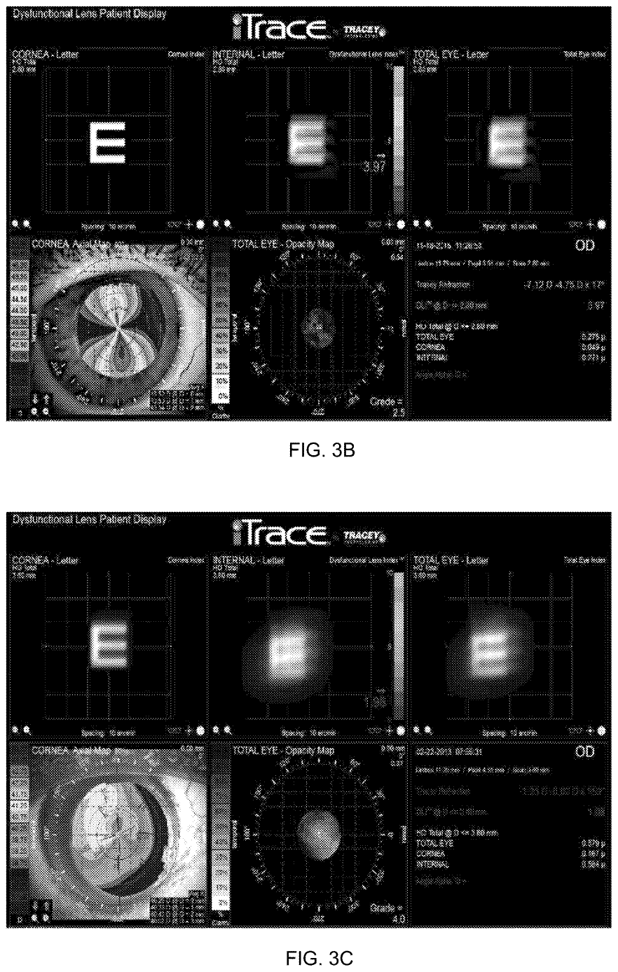

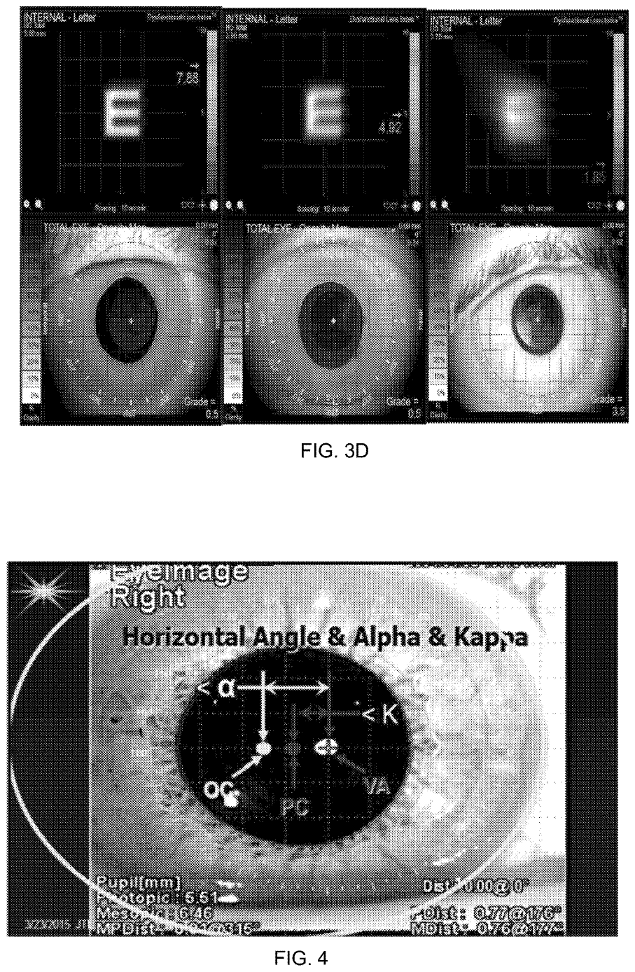

FIGS. 3A-3D show representative DLI patient displays. FIG. 3A shows a stage I early cataract in a 45 year old patient. FIG. 3B shows a stage II cataract with observable opacity and low DLI score. The cornea will support a toric Premium Lens. FIG. 3C shows a stage III cataract with an extremely low DLI score and high opacity grade (white area). The cornea is questionable with irregularities for optical support of a Premium lens. FIG. 3D is a DLI spectrum of aging lens stages I, II, and III.

### Optical Alignment Measurement

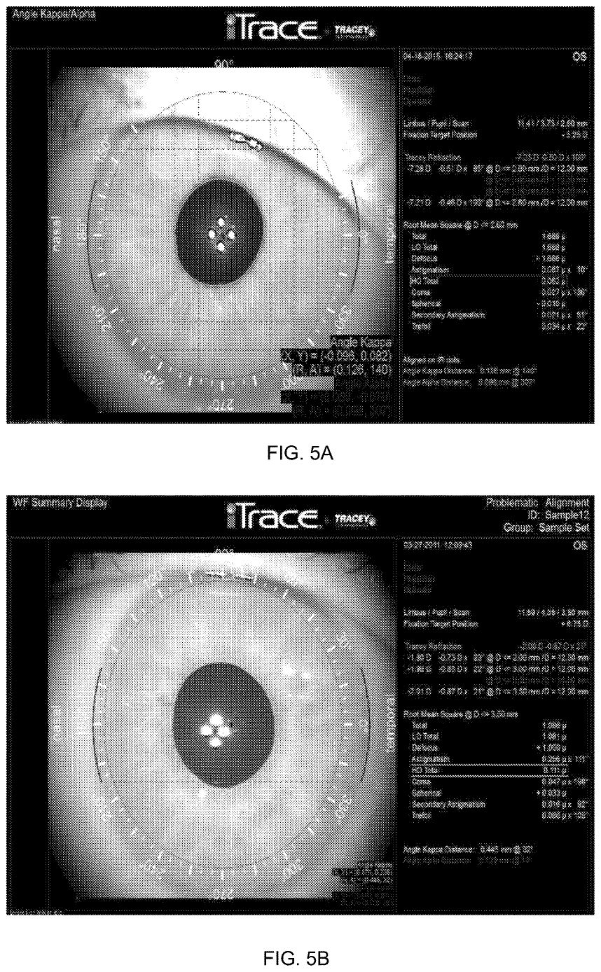

FIG. 4 shows the locations of the angle alpha (α) and angle kappa (κ) as the distances from the vertical axis (VA) to the optical center (OC) of the cornea and to the center of the pupil (PC), respectively. FIGS. 5A-5B are two cases that illustrate good optical alignment with low angle alpha in a first subject and an extremely high angle alpha in a second. Respectively, these subjects are a good candidate for multifocal intraocular lens (MIOL) and a poor candidate for MIOL.

### EXAMPLE 2

### Corneal Performance Index (CPI)

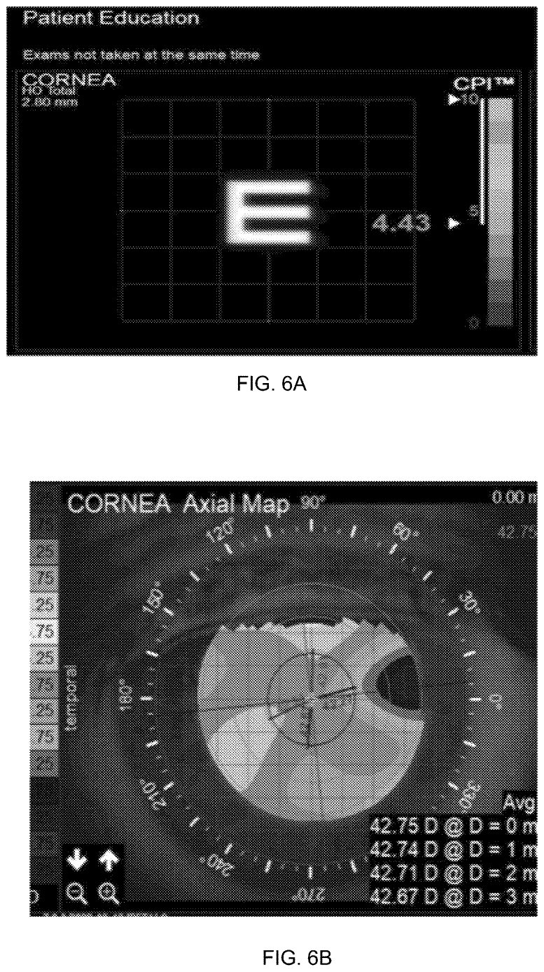

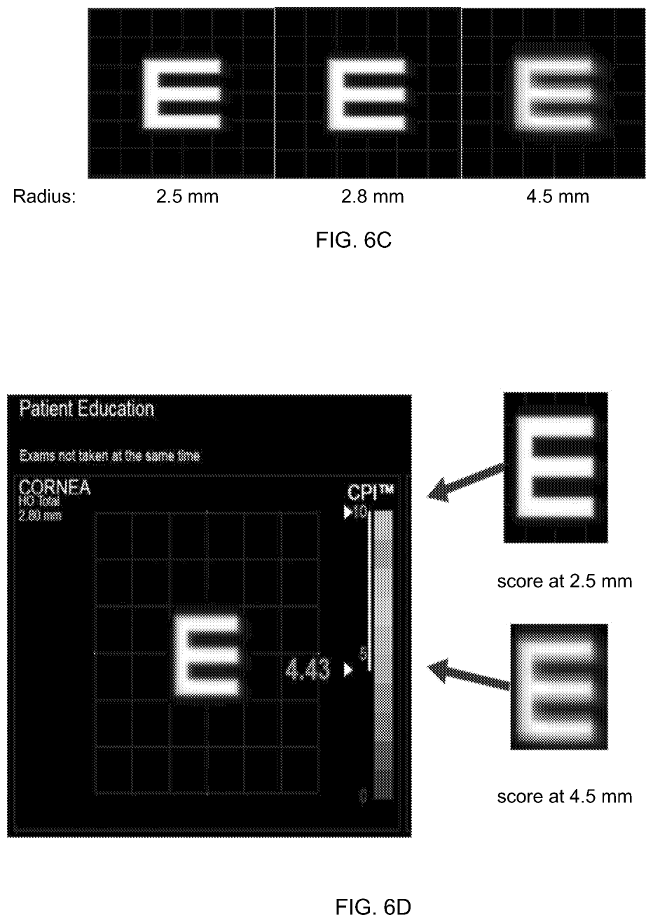

The Corneal Performance Index (CPI™) is a singular number that scores the quality of vision effects of the higher order aberrations generated at the cornea's anterior surface (FIG. 6A), which is a critical contribution to the patient's overall vision. The cornea's optical power (including the tear film on the anterior surface of the cornea) generates the majority of the refractive (focusing) power of the eye. This quality of vision index for the cornea is on a 0 to 10 scale with a bar that displays how that score would adjust based on the pupil size; for example, from 2.50 mm to 4.00 mm, or for any chosen pupil size typically up to 6.0 mm in diameter. The CPI number is automatically calculated from the cornea's higher order aberrations and is typically for the larger pupil sizes (4-6 mm) representing nighttime vision as this generally is the worst-case scenario with the lowest CPI as 10 is ideal for perfect vision. However, the CPI, if so chosen, can also be affected by the optical alignment between the cornea pupil and natural crystallin lens as this can be critical for determining premium IOL candidacy and selecting IOL types, such as multifocal, toric, aspheres or monofocals. The CPI creates a new standard in assisting eye care practitioners to determine if custom procedures are needed such as LASIK or special contact lenses to provide the patient with the best possible Quality of Vision after normal spectacle correction of spherical and cylindrical refractive error.

Corneal performance index grades the optical quality of the cornea by analyzing high order aberrations that degrades optical quality, it factors in optical alignment and pupil size as well for day and night analysis (FIG. 6A). The CPI is a Quality of Vision index by looking at different radii and get the corneal performance pertaining to that radius (FIG. 6B). The CPI takes the corneal topography, crops it at different radii between 2.5 to 4.5 mm, then calculates a score for each one of these points (FIG. 6C). The CPI attempts to address this by looking at different radii and get the corneal performance pertaining to that radius (FIG. 6D).

### EXAMPLE 3

### Quality of Vision Index

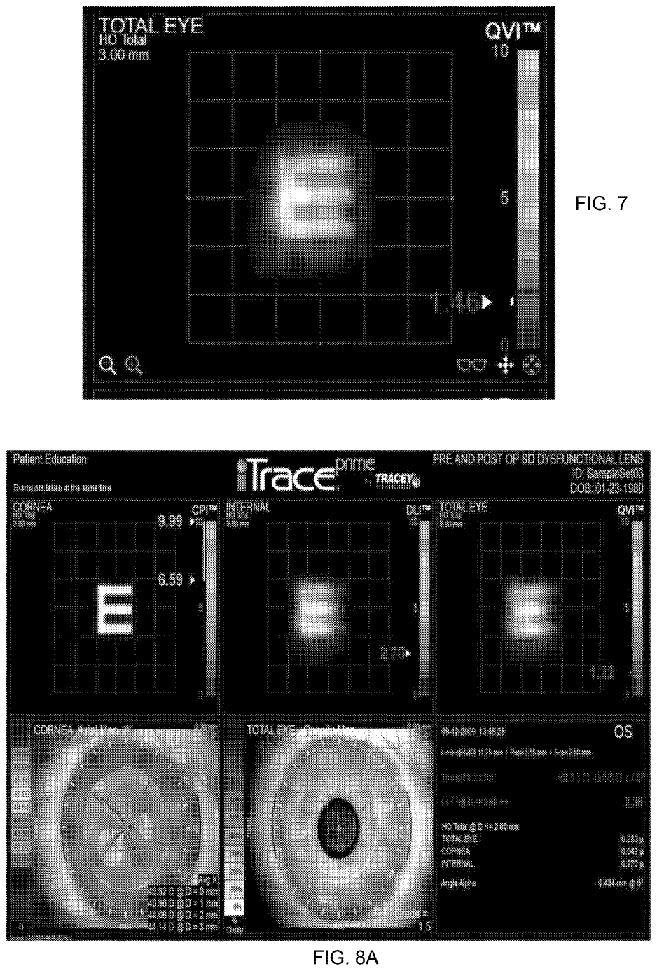

The Quality of Vision Index (QVI) is a number from 0 to 10 that scores the quality of vision for the total eye utilizing the higher order aberrations that are measured by ray tracing or wavefront technologies at the retinal plane. So this QVI does not involve the upper level processing of the visual cortex in the vision process as it is strictly based on the optical properties of the eye in generating an image at the retinal plane. As such the QVI utilizes only high order aberration information to generate its score. This Quality of Vision index is designed to correlate with patient's vision satisfaction after wearing standard optical correction; such as standard spectacles or eyeglasses correcting low order refractive errors, which are spherical errors in the common case of myopia and hyperopia and with or without astigmatism that would require cylinder correction. The QVI is a 0 to 10 score for visual performance to identify with patient complaints that today are not met through the typical 20/20 Snellen Visual Acuity standard. Many patients are 20/20 but unhappy with their vision as they still have some blur or halos, etc. The QVI, or any other namesake such as a WVI, is designed to objectively quantify such visual function disturbances patients experience every day or night with and without spectacle correction providing for a new Quality of Vision standard of care. The QVI quantifies the total optical performance of the total eye as it pertains to the optical image focused onto the retinal plane, primarily the macula, and may be selected for specific pupil sizes or for an overall value (FIG. 7).

### EXAMPLE 4

### Lens and Corneal Pathologies and Postoperative Problems

### Examples of Lens Pathologies in 3 Subjects

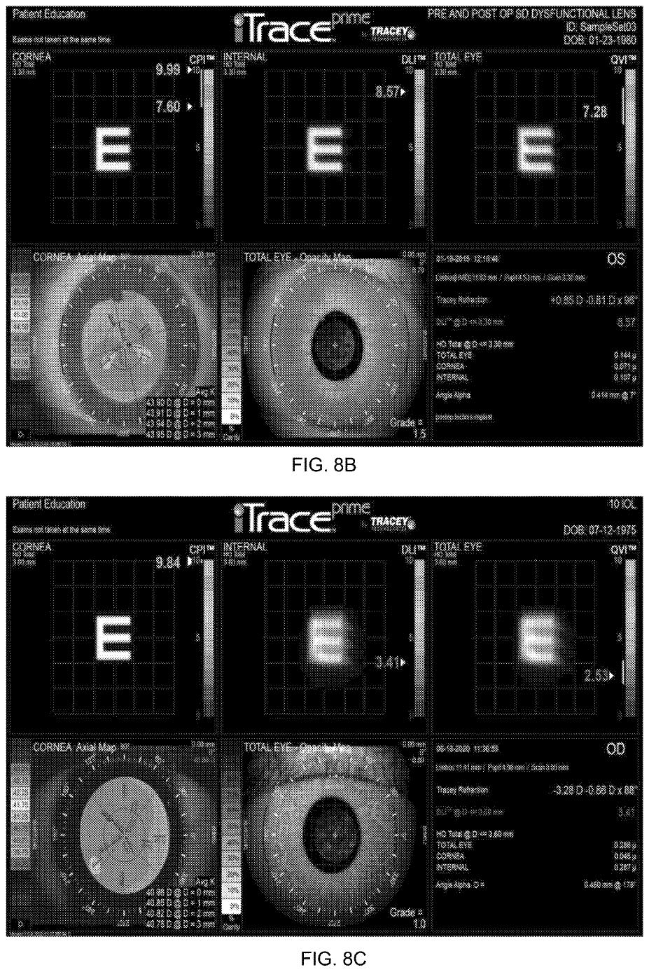

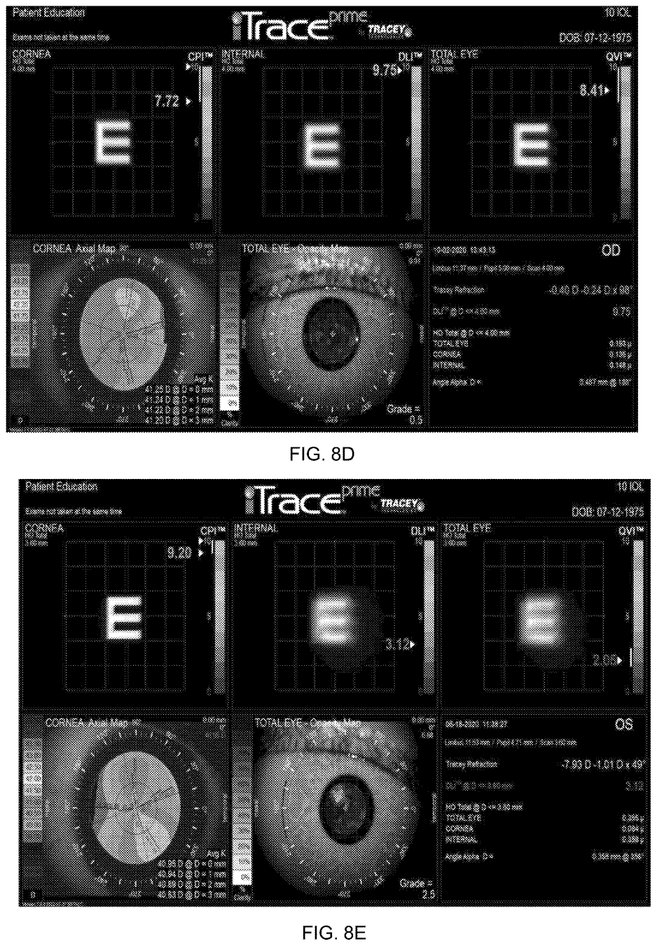

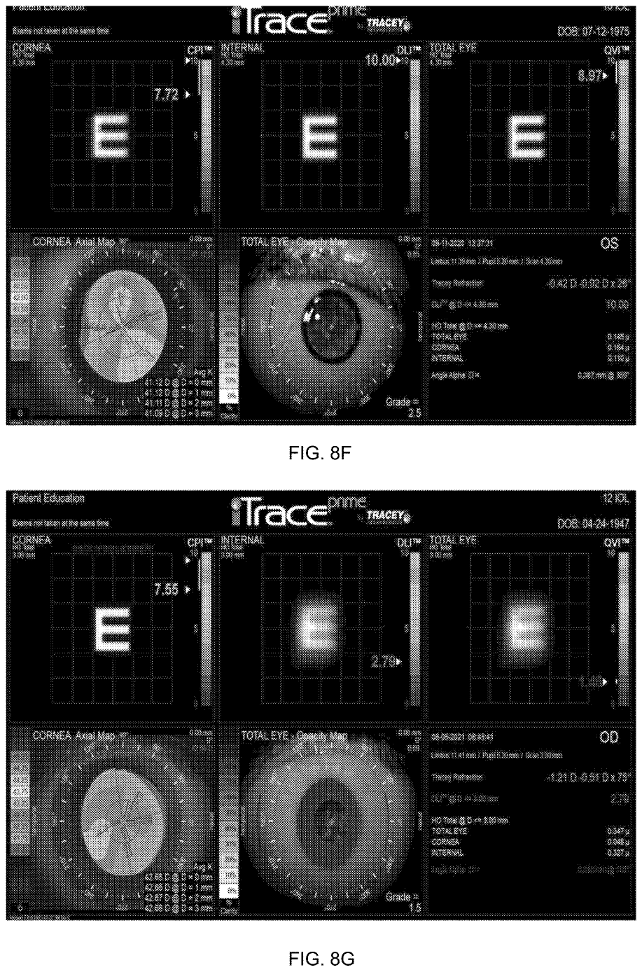

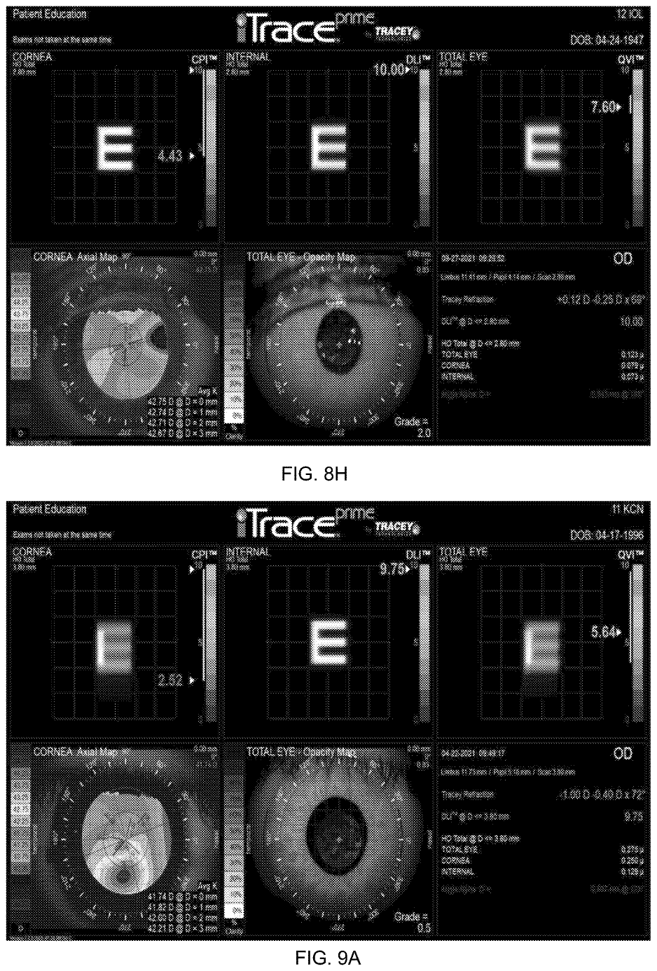

In case 1 the left eye (OS) is an example of a dysfunctional lens (FIG. 8A). There are no opacities on a slit lamp examination, but the patient cannot see well. FIG. 8B shows the postoperative improvement in the same patient.

In case 2 the right eye (OD) shows a preoperative early onset cataract (FIG. 8C) and the cataract postoperative with a Symfony ZXR00 intraocular lens (FIG. 8D) in the patient. The left eye early onset cataract in the patient is shown preoperatively in FIG. 8E and postoperatively with a Tecnis Multifocal ZLB00 in FIG. 8F.

In case 3 the preoperative patient has a cataract and treated dry eye (Keratoconjunctivitis sicca) (FIG. 8G) and postoperatively has a Symfony ZXR00.

### Examples of Corneal Pathologies in 3 Subjects

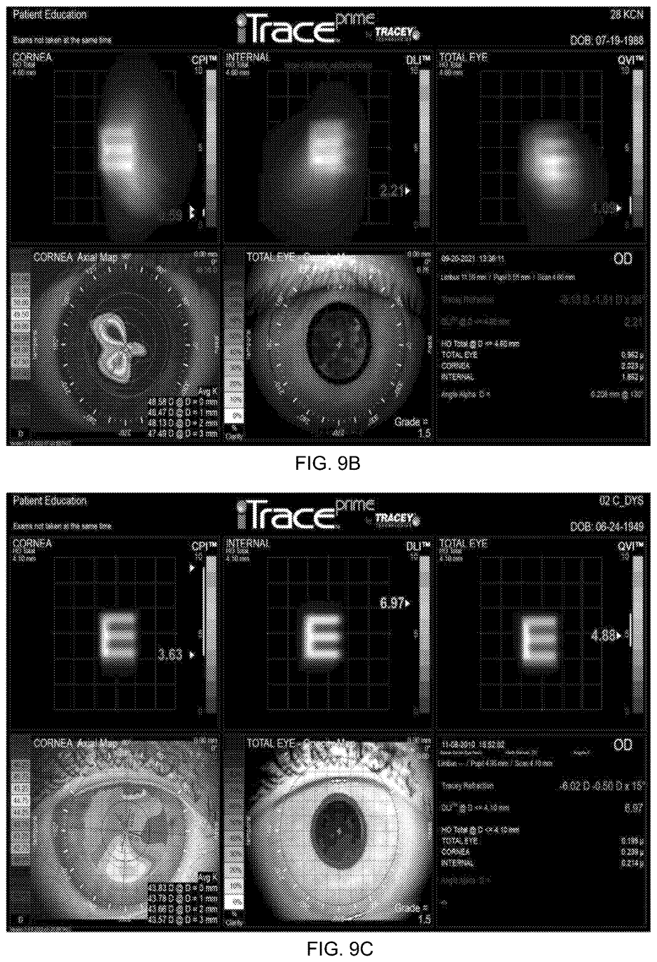

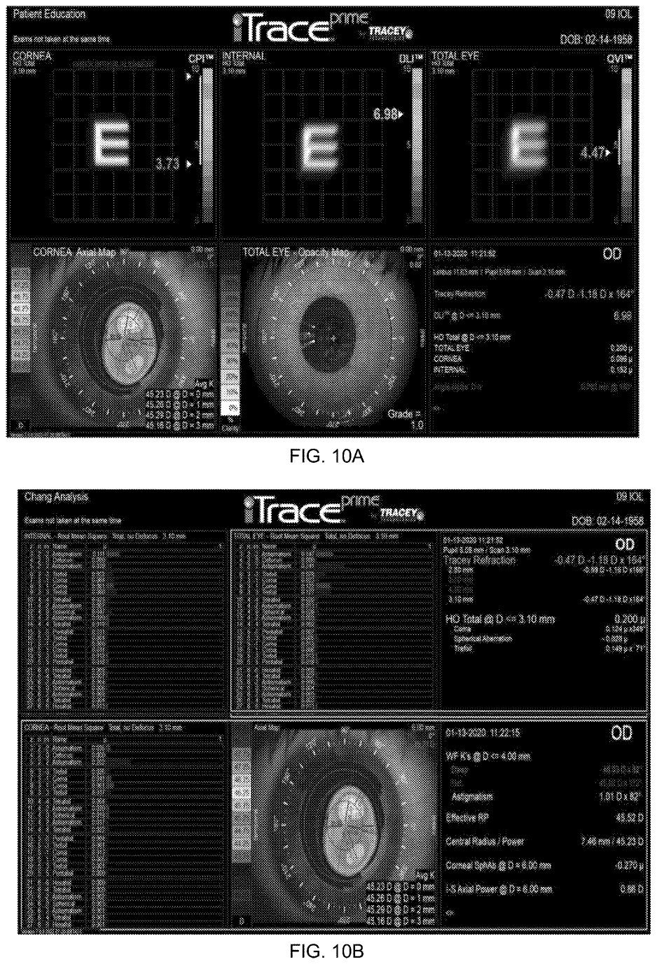

In case 4 a patient is keratoconic with moderate corneal and total vision performance (FIG. 9A).

In case 5 a patient is keratoconic with poor corneal and total vision performance (FIG. 9B).

In case 6 a patient has Reiss Buckler corneal dystrophy (FIG. 9C).

### Troubleshooting Post Operative Problems

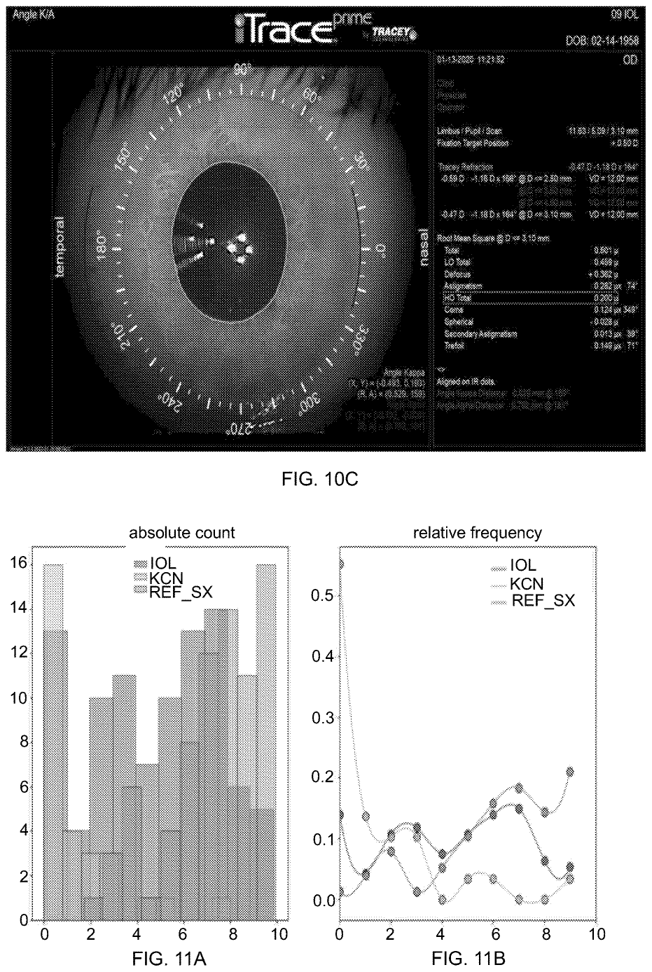

In case 7 a patient with hyperopic ablation undergoes refractive lens exchange with a Tecnis Premium ZLB00 IOL. Postoperatively the total eye performance is not great with little variation between day and night. The problem is that the aberrations in the cornea and lens are adding to each other (FIG. 10A). A closer look at the aberrations profile shows that the lens is adding to the problem of the total vision of this patient. The two main issues with the total vision are astigmatism and coma (FIG. 10B). The patient's optical alignment at least in part explains the coma and internal cylinder which is seen in this patient given that the patient had a non toric IOL (FIG. 10C).

CPI, DLI and QVI provide quality of vision solutions. They quantify the “intangible” aberrations of the eye that impact and reduce QOV, demonstrates when lens optics “compensates or decompensates” the cornea optics or when both are additive, and correlates to patient vision satisfaction and provides for predictive analytics to select best vision correction treatment and when to customize to optimize QOV.

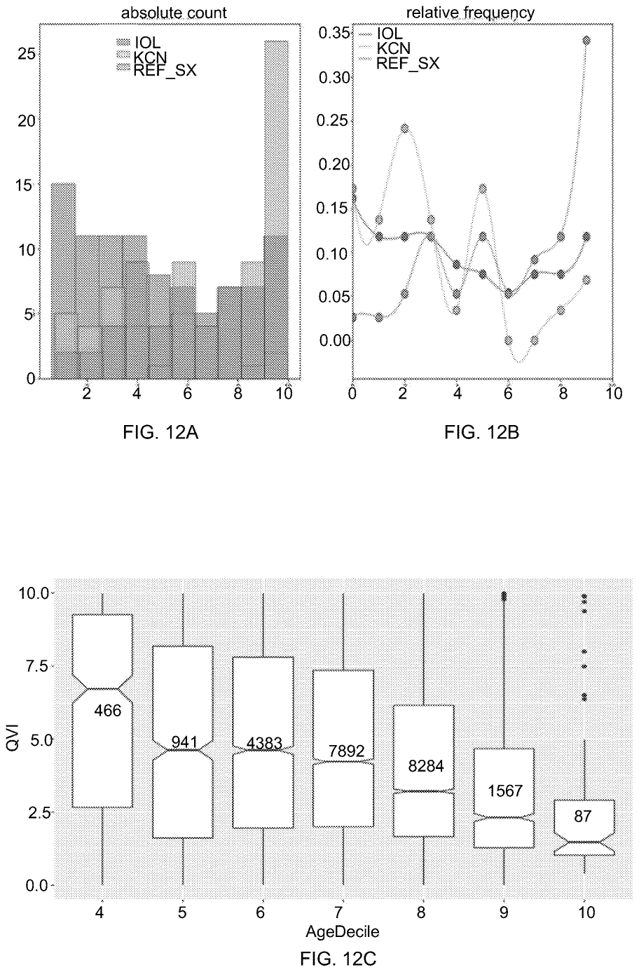

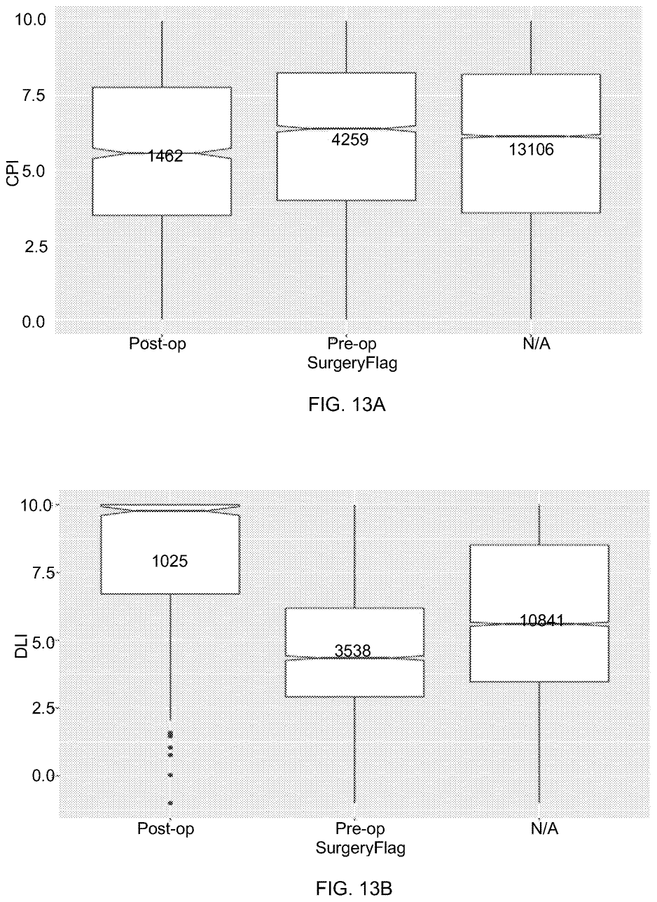

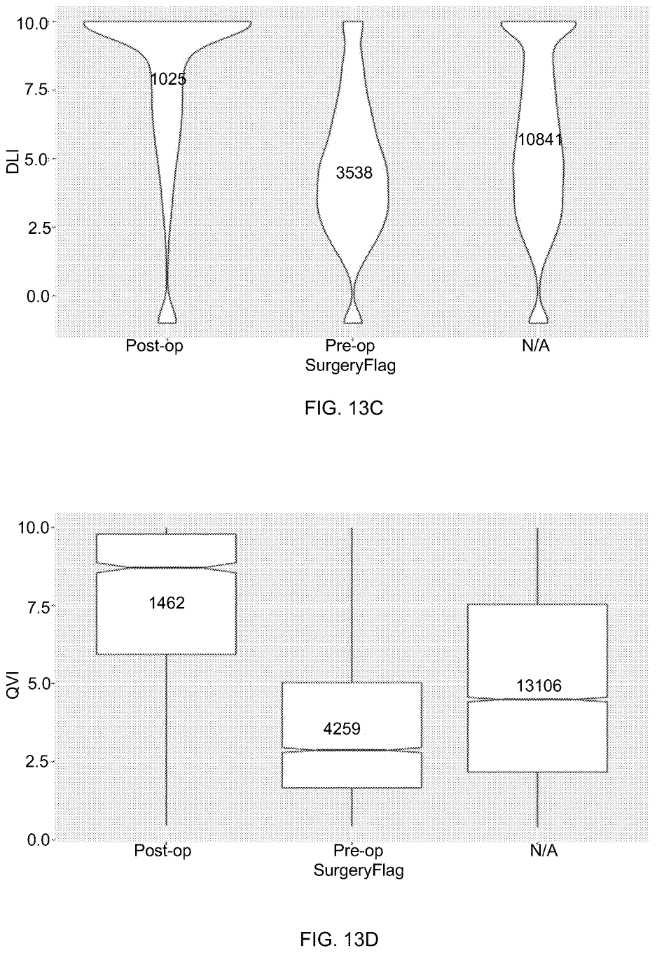

How these indices perform in different populations was determined. The performance of CPI in different populations is shown in FIGS. 11A-11B and that of QVI in FIGS. 12A-12B including illustrating the distribution of the QVI scores according to age deciles (FIG. 12C). In addition the scores for each of the CPI (FIG. 13A), DLI (FIGS. 13B-13C) and QVI (FIG. 13D) indices are distributed among postoperative, preoperative and no surgery populations.

### EXAMPLE 5

### Tear Function Index (TFI)

The Tear Function Index is a comprehensive index, with a single number score provided between 0 and 10, quantifying tear film quality and stability as it relates to visual function. A score of 10 is ideal as to provide excellent vision quality since the tear is a provider of optical surface performance of the cornea.

Besides tear film quality, TFI considers:
1. Spatial distribution of tear film quality (Central tear film changes within 4 mm are located within the pupil is more important for vision) and a two-dimensional map utilizing colors, as desired, can be generated over the cornea's surface for visualization.
2. Tear film dynamics: Stability over time or time to peak stability after a blink can incorporated into the TFI score

The TFI utilizes analysis of the Placido image off of the cornea surface but can also include other measures of optical or physical properties of the eye including assessment of tear film volume and/or in evaluating levels of aqueous or lipid content.

New tear film analysis software is used with the Tear Film Index (TFI) to help diagnose ocular surface disease and to analyze the vision quality effect. The goal is to differentiate between corneal irregular astigmatism and ocular surface disease and to guide the doctor and patient to the best treatment protocol.

### Tear Film Analysis With Placido Images

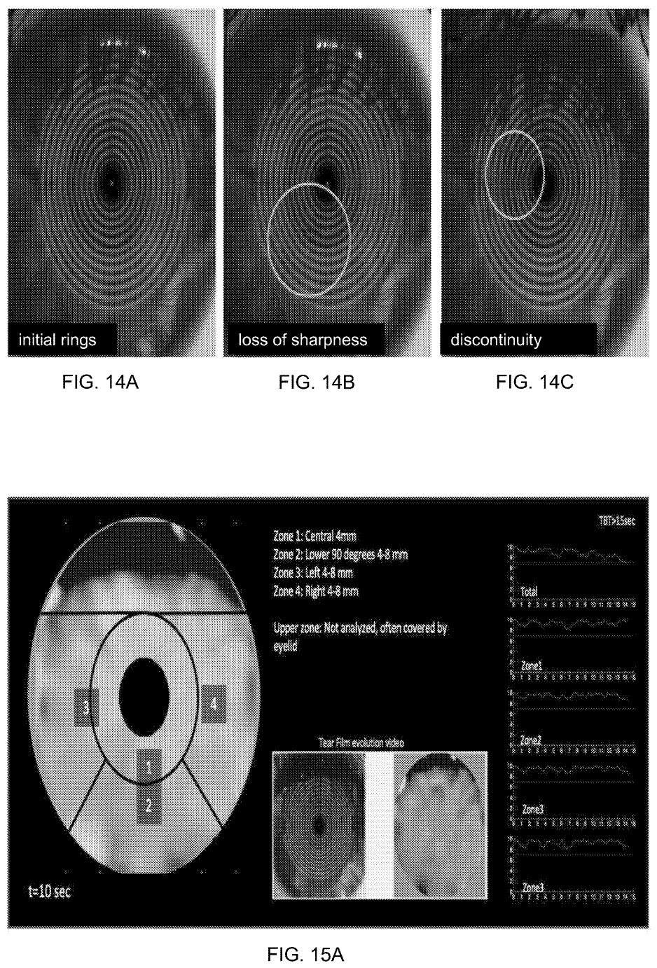

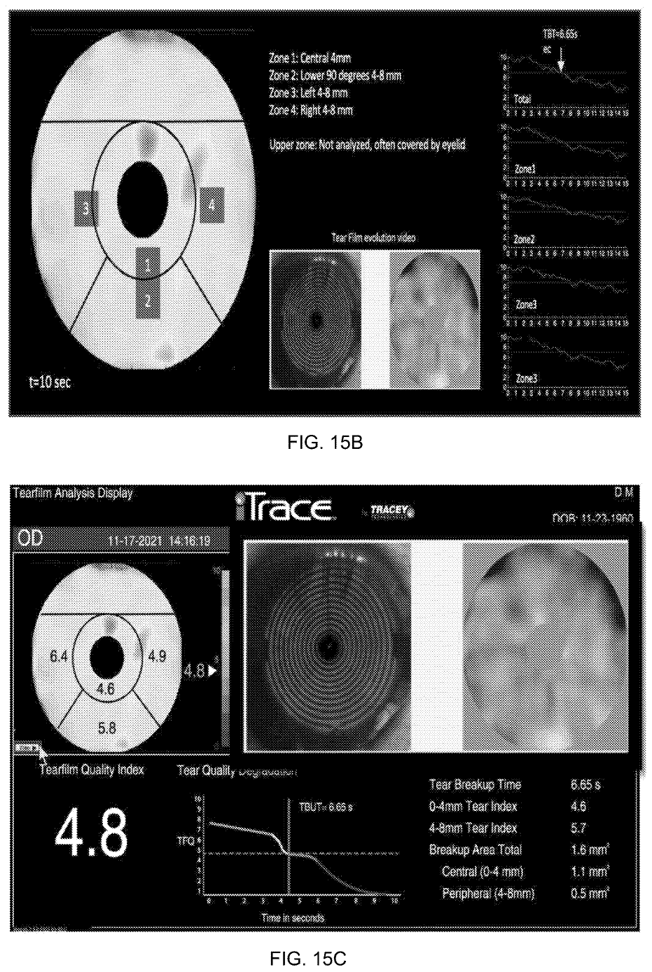

FIG. 14A shows a Placido ring. Analysis of a sequence of Placido images detects ring sharpness (FIG. 14B) and discontinuity (FIG. 14C). The TFI with Placido images provides an objective measurement of ring quality over time using Fourier domain analysis, an objective topographic tear breakup time and an automatic focus/alignment verification and blink rejection. FIG. 15A shows a normal tear film and FIG. 15B shows a film of a dry eye. A tear film analysis display is shown in FIG. 15C.

What is claimed is:

1. A method for predictive analytics of the quality of vision of a patient prior to a medical procedure, comprising:
measuring pre-operationally the optometric characteristics of the total eye and its components and spatial distributions and time variations thereof;
building a pre-operational digital twin based on measured optometric characteristics, said pre-operational digital twin consisting of a modifiable part and of a non-modifiable part;
building a pre-operational vector space from the modifiable part of said pre-operational digital twin and providing each vector therein with a correctable weight coefficient;
building pre-operationally a scoring metrics of the quality of vision represented by a quality of vision index (QVI), a cornea performance index (CPI), and a dysfunction lens index (DLI), each index containing related descriptors consisting of modifiable parts and non-modifiable parts of said pre-operational digital twin;
performing a comparative analysis of the pre-operational vector space with post-operational vector spaces acquired in the same medical procedures of other patients stored in a database, said comparative analysis defining the best fit vector space for the medical procedure;
calculating scoring metrics of the quality of vision based on measured and predicted data, said scoring metrics represented by the quality of vision index (QVI), the cornea performance index (CPI), and the dysfunction lens index (DLI);
modifying correctable weight coefficients of said best fit vector space to maximize related scoring metrics and presenting it as a predicted score for the medical procedure; and
reconstructing the pre-operational digital twin into a predicted digital twin by combining the non-modifiable part of the pre-operational digital twin and the modified part of the digital twin derived from the best fit vector space after its modification by the correctable weight coefficients.

2. The method of claim 1, further comprising:
storing said pre-operational digital twin and said predicted digital twin; and
displaying predicted data in a scoring metrics format and in a digital twin format.

3. The method of claim 2, further comprising:
repeating post-operationally the measuring step and the building steps;
performing post-operationally a comparative analysis of the predicted and of the current post-operational vector spaces in a machine-learning procedure correcting the weight coefficients for the same medical procedures in the future, and
displaying the results of post-operational machine-learning comparative analysis, storing the results of post-operational machine-learning comparative analysis.

4. The method of claim 3, wherein the machine learning is based on k-nearest neighbours (kNN) or on neural networking.

5. The method of claim 1, wherein calculating said refraction quality index is based on a comparison of a reference mesh of nodes created from coordinates of beam-projected points at an eye aperture with the mesh of nodes representing said coordinates of said beam-projection points in a plane of the eye aperture modified by shifts in a plane of a retina by a refraction state in the beam-projection points, comprising:
a) measuring a shape distortion of each cell of said mesh of nodes,
b) identifying each cell by a degree of distortion,
c) building a histogram of distortion spectrum,
d) defining a distortion reference threshold for RQI calculation, and
e) calculating RQI as a number of over-threshold shape-distorted cells to a total number of cells in the mesh.

6. The method of claim 1, wherein calculating the quality of vision index (QVI) to score the quality of vision of the total eye is based on an impact of higher order aberrations on a point spread function of the total eye under standard measurement conditions with an empirically corrected individual impact of each of said higher order aberrations.

7. The method of claim 1, wherein calculating the cornea performance index (CPI) to score the corneal fraction in the quality of vision is based on an analysis of a wave front reconstructed from a corneal topography and conditions of tear film dynamics.

8. The method of claim 1, wherein calculating the dysfunction lens index DLI to score the quality of the internal optics of an eye is based on a contribution of higher order aberrations measured for the internal optics either by substracting a wave front calculated for a cornea from a wave front of a total eye, or by measuring separately a fraction of the aberrations produced by the internal optics.

9. The method of claim 1, wherein the quality of vision index (QVI), the cornea performance index (CPI) and the dysfunction lens index (DLI) have a decimal 0 to 10 scale, where the lowest quality is scored by 0, and the highest quality is scored by 10.

10. The method of claim 1, wherein the optometric characteristics comprise a refractive part measured by ray tracing and a structural part measured by optical coherence tomography.

11. A system for predictive analytics of the quality of vision, comprising:
a sensor subsystem containing a first sensor, a second sensor and a third sensor configured to measure, separately or in combination, optometric characteristics of a total eye and its components, their spatial distributions and time variations;
a storage subsystem consisting of a preoperational memory, an archived-cases memory, and a dynamic interrogation switch, said preoperational memory and said archived-cases memory consisting of per-patient cells each of which has a preoperational sub-cell, a prediction sub-cell, and a post-operational sub-cell, and each of said per-patient cell in the archived-cases memory connected to the dynamic interrogation switch;
a control subsystem containing a control signals generator, an interface, and a display, said display connected to the decision-making subsystem through the interface and said control signals generator connected to the interface;
a decision-making subsystem consisting of a twin builder, a first metrics builder, a second metrics builder, a first builder of vector space, a second builder of vector space, a processing unit with a first input and a second input, and a weight coefficients unit, said first builder of vector space connected to the first input, said second builder of vector space connected to the second input and outputs of said first metrics builder and of said second metrics builder are connected to the display;
an intra-system communication bus configured to exchange signals, commands and data among the sensor subsystem, the decision-making subsystem, the storage subsystem, and the control system pre-operationally and post-operationally.

12. The system of claim 11, wherein, pre-operationally, in the decision-making subsystem:
a) the twin builder is configured to:
generate a pre-operational digital twin of vision in an eye of a patient by integration of data from the sensor subsystem, said digital twin consisting of a first part not modified by a medical procedure and a second part subject to modification by a medical procedure;
deliver both the first part of the digital twin and the second part of the digital twin to the first metrics builder and to the storage subsystem; and
deliver the second part of the digital twin to the first builder of vector space;
b) the first metrics builder is configured to generate a score for quality of vision from a quality of vision index (QVI), a dysfunction lens index (DLI), and a cornea performance index (CPI), each of said OVI, DLI and CPI indices is configured to integrate current data from the sensor subsystem;
c) the second builder of vector space is configured to receive digital twins from the archived-cases memory consecutively in time through the dynamic interrogation switch, said digital twins delivered to the processing unit under control of the intra-system communication bus;
d) the processing unit is configured to:
analyze vector spaces received from the first builder of vector space and the second builder of vector space and to generate an output for decision making and for machine-human communication and interaction; and
deliver a modified part of a predicted digital twin to the second metrics builder and to the storage subsystem, said first part of the digital twin being delivered from the twin builder;
e) the second metrics builder is configured to generate a score for quality of vision from a quality of vision index (QVI), a dysfunction lens index (DLI), and a cornea performance index (CPI), each of said OVI, DLI and CPI indices is configured to integrate data from an output of the processing unit; and
f) a current-case cell with its two clusters filled-in by the preoperational twin and the prediction twin is configured to be repositioned from said preoperational memory to said archived-cases memory to keep the post-operational sub-cell free for filling-in after the machine-learning step.

13. The system of claim 12, wherein, post-operationally, in the decision-making subsystem:
g) the twin builder is configured to:
generate a post-operational digital twin of the vision of the patient by integrating data from said sensor subsystem, said post-operational digital twin consisting of a first part not modified by a medical procedure and a second part modified by a medical procedure;
deliver both the first part and the second part of the post-operational digital twin to the first metrics builder and to the storage subsystem; and
deliver the second part of the digital twin to the first builder of vector space;
h) the first metrics builder is configured to generate the score for quality of vision from the quality of vision index (QVI), the dysfunction lens index (DLI), and the cornea performance index (CPI), each of said OVI, DLI and CPI indices is configured to integrate current data from the sensor subsystem;
i) the second builder of vector space is configured to receive the digital twins from the archived-cases memory consecutively in time through the dynamic interrogation switch, said digital twins delivered to the processing unit under control of the intra-system communication bus;
j) the processing unit is configured to:
analyze vector spaces received from the first builder of vector space and the second builder of vector space and to generate an output for decision making and for machine-human communication and interaction;
deliver a modified part of a post-operative digital twin to the second metrics builder and to the storage subsystem to fill-in a post-operative cluster of the archived cases cells, said first part of the post-operative digital twin delivered to the second metrics builder from the twin builder; and
apply the machine-learning algorithm to correct a weight of each vector in the vector space for the quality of vision prediction for the next patient to minimize a difference between the predicted metrics and the post-operational metrics; and
k) the second metrics builder is configured to generate a post-operative score for the quality of vision from the quality of vision index (QVI), the dysfunction lens index (DLI), and the cornea performance index (CPI), each of said OVI, DLI and CPI indices is configured to integrate data from the output of the processing unit.

14. The system of claim 13, wherein the machine-learning algorithm is applied as part of an artificial intelligence system.

15. The system of claim 11, wherein the output of the second metric builder is displayable before and after application of the machine-learning algorithm.

16. The system of claim 11, wherein the first sensor is a wave front aberrometer, the second sensor is a corneal topographer and the third sensor is an optical coherence tomograph.

17. The system of claim 11, wherein the first sensor is a wave front aberrometer configured for ray tracing to measure synchronously aberrations of the optometric characteristics of:
a) aberrations of the total eye and of the cornea,
b) intraocular distances,
c) tear film dynamics, and
d) optical dysfunction of the crystalline lens.

18. The system of claim 11, wherein the first sensor, the second sensor and the third sensor are integrated optically to have a common optical axis and mechanically as a solid construction.

19. The system of claim 11, wherein the sensor subsystem, the control subsystem and the decision-making subsystem are configured in at least one three-part combination wirelessly connected to the storage subsystem, said storage subsystem residing in the cloud.

20. The system of claim 19, wherein each of the at least one three-part combination has an exchange-communication modem and the storage subsystem has an exchange-synchronization modem configured to control each of said three-part combinations independently of each other with a data flow synchronized by the exchange-synchronization modem.

21. The system of claim 11, wherein the sensor subsystem and the control subsystem are configured as at least one first two-part combination and the decision-making subsystem and the storage subsystem are configured as a second two-part combination; wherein the at least one first two-part combination is connected wirelessly to the second two-part combination, said second two-part combination residing in the cloud.

22. The system of claim 21, wherein each of the at least one two-part combination has an exchange-communication modem and the second two-part combination has an exchange-synchronization modem; wherein each of the first two-part combinations is controlled independently of each other with a data flow synchronized by the exchange-synchronization modem.
